(19) United States
(12) Patent Application Publication
(10) Pub. No.: US 2022/0372171 A1
(43) Pub. Date: Nov. 24, 2022

(54) ANTIBODY DIRECTED AGAINST TENOFOVIR AND DERIVATIVES THEREOF

(71) Applicants: Abbott Rapid Diagnostics International Unlimited Company, Dublin (IE); The Regents of the University of California, Oakland, CA (US)

(72) Inventors: Michael Vincent, Pomona, CA (US); Warren Rodrigues, Pomona, CA (US); Monica Gandhi, San Francisco, CA (US); Guohong Wang, Claremont, CA (US)

(21) Appl. No.: 17/761,928

(22) PCT Filed: Sep. 18, 2020
(86) PCT No.: PCT/US2020/051580

§ 371 Date: Mar. 18, 2022

Related U.S. Application Data
(60) Provisional application No. 62/903,404, filed on Sep. 20, 2019.

(51) Int. Cl. C07K 16/44; G01N 33/94

(52) U.S. Cl. CPC C07K 16/44; G01N 33/94

(57) ABSTRACT

The disclosure is directed to a polyclonal antibody composition comprising a heterologous population of mammalian antibodies capable of specifically binding to tenofovir or a tenofovir derivative in a sample. Methods and assays for detecting tenofovir or a tenofovir derivative in a sample using the polyclonal antibody composition also are provided.

### CROSS-REFERENCE TO RELATED APPLICATIONS

This application claims the benefit of U.S. Provisional Patent Application No. 62/903,404, filed Sep. 20, 2019, the contents of which are incorporated by reference herein.

### FIELD

The present invention relates to antibodies directed against tenofovir and tenofovir derivatives, as well as compositions and kits comprising same.

### BACKGROUND

Tenofovir (TFV) is a nucleotide reverse transcriptase inhibitor that selectively inhibits the reverse transcriptase (RT) enzyme in retroviruses like HIV-1 and hepatitis B. It is used primarily in the treatment of HIV-1/AIDS and chronic Hepatitis B infections. Tenofovir induces premature chain termination of DNA transcription by incorporation into a growing DNA strand, thereby preventing viral replication and reducing viral load. “PrEP” (Pre-Exposure Prophylaxis) therapy refers to a daily regimen of tenofovir and emtricitabine to prevent HIV infection. Daily doses of tenofovir have been shown to cause a 48.9% reduced incidence of HIV in subjects who are at high risk for infection through sexual transmission and drug use.

Pharmacologic measures of adherence to tenofovir disoproxil fumarate (TDF)/emtricitabine (FTC)-based PrEP, where TFV drug levels are assessed in a matrix such as plasma, dried blood spots (DBS), or hair (see, e.g., Gandhi, M. and Greenblatt, R. M., Ann Intern Med., 137(8): 696-697 (2002); Gandhi et al., AIDS, 23(4): 471-478 (2009); and Liu et al., PLoS One, 9(1): e83736. 3885443 (2014)), capture drug ingestion and predict outcomes more accurately than self-reported adherence (Marrazzo et al., N Engl J Med, 372(6):509-518 (2015); Grant et al., N Engl J Med, 363(27): 2587-2599 (2010); Anderson et al., Sci Transl Med., 4(151): 151ra125 (2012); Van Damme, L. and Corneli, A., N Engl J Med., 368(1): 84 (2013); Blumenthal, J. and Haubrich, R., Expert Opin. Pharmacother., 14(13): 1777-1785 (2013); Musinguzi et al., AIDS, 30(7):1121-1129 (2016); Donnell et al., J Acquir Immune Defic Syndr., 66(3): 340-348 (2014); and Thigpen et al., N Engl J Med., 367(5): 423-434 (2012)). Pharmacologic adherence monitoring is especially important in PrEP (Baxi et al., J Acquir Immune Defic Syndr., 68(1):13-20 (2015); and Koss et al., Clin Infect Dis., 66(2): 213-219 (2018)), where a surrogate biomarker of response (e.g., HIV viral loads as in treatment) is not available. Current methods to analyze PrEP drug levels in any matrix, however, including easily accessible urine (Koenig et al., HIV Med., 18(6): 412-418 (2017)), require liquid chromatography-tandem mass spectrometry (LC-MS/MS), which cannot be performed in real-time.

There remains a need for compositions and methods that provide precise, rapid, and low-cost monitoring of adherence to PrEP therapy with tenofovir, or derivatives thereof.

### BRIEF SUMMARY OF THE INVENTION

The disclosure provides polyclonal antibody composition comprising a heterogeneous population of mammalian antibodies that specifically bind tenofovir (TFV) or a tenofovir derivative, wherein the heterogeneous population of mammalian antibodies is generated against a compound of formula (I) or formula (II):

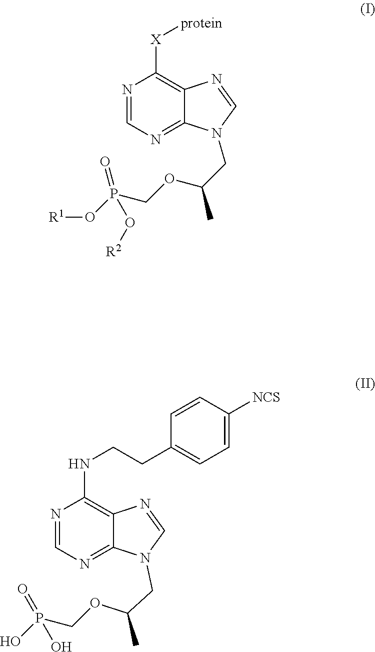

or a pharmaceutically acceptable salt thereof, wherein: R¹ and R² are each independently selected from the group consisting of hydrogen, C₁-C₆ alkyl, C₂-C₆ alkenyl, C₂-C₆ alkynyl, aryl, arylalkyl, cyanoalkyl, and —(C₁-C₆-alkylene)-Y—(C₁-C₆ alkyl), wherein Y is selected from —O—, —NH—, —S—, —C(O)NH—, —C(O)O—, —C(O)S—, —OC(O)NH—, —OC(O)O—, and —NHC(O)NH—; and X is a linker.

The disclosure also provides a solid support for detecting the presence of tenofovir or a tenofovir derivative in a sample, which comprises the aforementioned polyclonal antibody composition immobilized thereon.

Also provided is a method of detecting tenofovir or a tenofovir derivative in a sample obtained from a subject, which method comprises: (a) contacting a sample obtained from a subject with the aforementioned solid support under conditions which allow binding of tenofovir or a tenofovir derivative, if present in the sample, to the polyclonal antibody composition, and (b) detecting binding of tenofovir or a tenofovir derivative bound to the polyclonal antibody composition.

The disclosure further provides an assay for detecting the presence of tenofovir or a tenofovir derivative in a sample obtained from a subject, which comprises: (a) contacting a biological sample with the aforementioned polyclonal antibody composition, wherein the subject is undergoing treatment with tenofovir or a tenofovir derivative; and (b) detecting the polyclonal antibody composition bound to tenofovir or a tenofovir derivative.

The disclosure provides use of a polyclonal antibody composition to detect tenofovir or a tenofovir derivative in a sample obtained from a subject, wherein the polyclonal antibody composition comprises a heterogeneous population of mammalian antibodies that specifically bind tenofovir (TFV) or a tenofovir derivative, and wherein the heterogeneous population of mammalian antibodies is generated against a compound of formula (I) or formula (II):

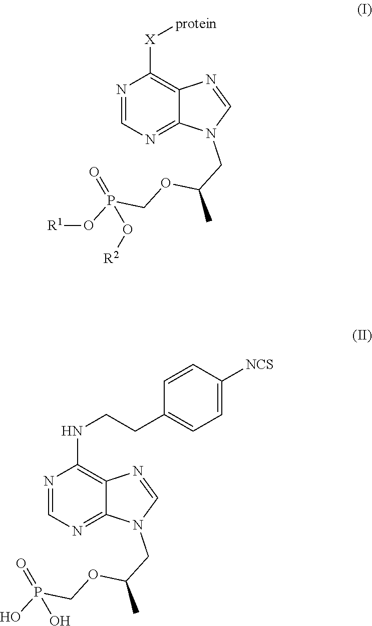

or a pharmaceutically acceptable salt thereof, wherein. R¹ and R² are each independently selected from the group consisting of hydrogen, C₁-C₆ alkyl, C₂-C₆ alkenyl, C₂-C₆ alkynyl, aryl, arylalkyl, cyanoalkyl, and —(C₁-C₆-alkylene)-Y—(C₁-C₆ alkyl), wherein Y is selected from —O—, —NH—, —S—, —C(O)NH—, —C(O)O—, —C(O)S—, —OC(O)NH—, —OC(O)O—, and —NHC(O)NH—; and X is a linker.

### BRIEF DESCRIPTION OF THE DRAWINGS

FIG. 1 is a table which sets forth data from ELISA and LFA immunoassays performed on urine samples from the Partners PrEP study. Both immunoassays utilized the tenofovir-binding antibody disclosed herein. The immunoassay data is compared to data from liquid chromatography-tandem mass spectrometry (LC-MS/MS) performed on plasma samples.

FIGS. 2A-2G are tables showing curve data for Partners PrEP urine samples 1-38 (FIG. 2A), 39-76 (FIG. 2B), 77-114 (FIG. 2C), 115-152 (FIG. 2D), 153-190 (FIG. 2E), 191-229 (FIG. 2F), and 230-250 (FIG. 2G).

FIG. 3 is a table which sets forth data from ELISA and LFA immunoassays performed on urine samples from the I-BrEATHe study. Both immunoassays utilized the tenofovir-binding antibody disclosed herein. The immunoassay data is compared to data from liquid chromatography-tandem mass spectrometry (LC-MS/MS) performed on plasma samples.

FIGS. 4A-4G are tables showing curve data for I-BrEATHe urine samples 1-38 (FIG. 4A), 39-76 (FIG. 4B), 77-114 (FIG. 4C), 115-152 (FIG. 4D), 153-190 (FIG. 4E), 191-228 (FIG. 4F), and 229-231 (FIG. 4G).

### DETAILED DESCRIPTION OF THE INVENTION

The present disclosure is predicated, at least in part, on the generation of a highly specific polyclonal antibody that binds to tenofovir (TFV) and tenofovir derivatives, which is capable of detecting TFV at clinically relevant cutoffs in urine and serum.

### Definitions

To facilitate an understanding of the present technology, a number of terms and phrases are defined below. Additional definitions are set forth throughout the detailed description.

The term “immunoglobulin” or “antibody,” as used herein, refers to a protein that is found in blood or other bodily fluids of vertebrates, which is used by the immune system to identify and neutralize foreign objects, such as bacteria and viruses. Typically, an immunoglobulin or antibody is a protein that comprises at least one complementarity determining region (CDR). The CDRs form the “hypervariable region” of an antibody, which is responsible for antigen binding (discussed further below). A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (x) or lambda (k), based upon the amino acid sequences of their constant domains. In a typical immunoglobulin, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). An antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H1} domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

The term “monoclonal antibody,” as used herein, refers to an antibody produced by a single clone of B lymphocytes that is directed against a single epitope on an antigen. In contrast, “polyclonal” antibodies are antibodies that are secreted by different B cell lineages within an animal. Polyclonal antibodies are a heterogenous collection of immunoglobulin molecules that recognize multiple epitopes on the same antigen.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, uracil, adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

The terms “immunogen” and “antigen” are used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal. By “epitope” is meant a sequence of an antigen that is recognized by an antibody or an antigen receptor. Epitopes also are referred to in the art as “antigenic determinants.” In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The antigen can be a protein or peptide of viral, bacterial, parasitic, fungal, protozoan, prion, cellular, or extracellular origin, which provokes an immune response in a mammal, preferably leading to protective immunity.

The terms “detectable label,” and “label,” as used herein, refer to a moiety that can produce a signal that is detectable by visual or instrumental means. The detectable label may be, for example, a signal-producing substance, such as a chromogen, a fluorescent compound, an enzyme, a chemiluminescent compound, or a radioactive compound. In one embodiment, the detectable label may be a fluorescent compound, such as a fluorophore.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

The term “alkyl,” as used herein, means a straight or branched saturated hydrocarbon chain containing from 1 to 24 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), or 1 to 4 carbon atoms (C₁-C₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

The term “alkenyl,” as used herein, refers to a straight or branched hydrocarbon chain containing from 2 to 24 carbon atoms and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkynyl,” as used herein, refers to a straight or branched hydrocarbon chain containing from 2 to 24 carbon atoms and containing at least one carbon-carbon triple bond. Representative examples of alkynyl include, but are not limited to, ethynyl, propynyl, and butynyl.

The term “aryl,” as used herein, refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C10 aryl”; e.g., naphthyl such as 1-naphthyl and 2-naphthyl). In some embodiments, an aryl group has fourteen ring carbon atoms (“C₁₄ aryl”; e.g., anthracenyl and phenanthrenyl). An aryl group may be described as, e.g., a C₆-C₁₄-membered aryl, wherein the term “membered” refers to the non-hydrogen ring atoms within the moiety. Aryl groups include, but are not limited to, phenyl, naphthyl, anthracenyl and phenanthrenyl.

The term “arylalkyl,” as used herein, means an alkyl group, as defined herein, in which at least one hydrogen atom (e.g., one, two, or three hydrogen atoms) is replaced by an aryl group. Representative examples of arylalkyl include, but are not limited to, benzyl, 2-phenylethyl, 3-phenylpropyl, 9-fluorenyl, benzhydryl, and trityl groups.

The term “cyano” refers to the radical —CN.

The term “cyanoalkyl,” as used herein, means an alkyl group, as defined herein, in which at least one hydrogen atom (e.g., one hydrogen atom) is replaced by a cyano group.

The term “cycloalkyl,” as used herein, refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

The term “heteroalkyl,” as used herein, means an alkyl group, as defined herein, in which one or more of the carbon atoms has been replaced by a heteroatom independently selected from S, O, P and N. Representative examples of heteroalkyls include, but are not limited to, alkyl ethers, secondary and tertiary alkyl amines, and alkyl sulfides.

The term “heteroaryl,” as used herein, refers to an aromatic monocyclic ring or an aromatic bicyclic ring system or an aromatic tricyclic ring system. The aromatic monocyclic rings are five or six membered rings containing at least one heteroatom independently selected from the group consisting of N, O, and S (e.g. 1, 2, 3, or 4 heteroatoms independently selected from O, S, and N). The five-membered aromatic monocyclic rings have two double bonds and the six membered six membered aromatic monocyclic rings have three double bonds. The bicyclic heteroaryl groups are exemplified by a monocyclic heteroaryl ring appended fused to a monocyclic aryl group, as defined herein, or a monocyclic heteroaryl group, as defined herein. The tricyclic heteroaryl groups are exemplified by a monocyclic heteroaryl ring fused to two rings independently selected from a monocyclic aryl group, as defined herein or a monocyclic heteroaryl group as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazolyl, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, thiazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

The term “heterocycle” or “heterocyclic” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings.

As used herein, the terms “alkylene,” “arylene,” “heteroalkylene,” “heteroarylene,” “cycloalkylene,” and “heterocyclylene” mean a divalent radical derived from an alkyl, aryl, heteroalkyl, heteroaryl, cycloalkyl, or heterocyclyl group, respectively.

In some instances, the number of carbon atoms in a group (e.g., alkyl) is indicated by the prefix “Cₓ-C_{y}-”, wherein x is the minimum and y is the maximum number of carbon atoms in the group. Thus, for example, “C₁-C₃-alkyl” refers to an alkyl group containing from 1 to 3 carbon atoms.

The term “substituent” refers to a group substituted on an atom of the indicated group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable group known to those of skill in the art (e.g., one or more of the groups recited below). Substituent groups include, but are not limited to, halogen, keto, thio, cyano, isocyano, thiocyano, isothiocyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, aryloxy, amino, alkylamino, acylamino, aminoalkyl, arylamino, sulfonylamino, sulfinylamino, sulfonyl, alkylsulfonyl, arylsulfonyl, aminosulfonyl, sulfinyl, carboxyl, ketone, amide, carbamate, thiocarbamate and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### Immunogen

As discussed above, tenofovir (TFV) is a nucleotide analog reverse-transcriptase inhibitor (NtRTI). Tenofovir lacks a hydroxyl group in the position corresponding to the 3′ carbon of the d-AMP, preventing the formation of the 5′ to 3′ phosphodiester linkage essential for DNA chain elongation. Once incorporated into a growing DNA strand, tenofovir causes premature termination of DNA transcription, preventing viral replication. Tenofovir disoproxil fumarate (tenofovir DR, TDF) is marketed in the U.S. as VIREAD® by Gilead and is approved for the treatment of HIV infection and chronic hepatitis B virus (HBV) infection in adults and children. Tenofovir is also available in fixed-dose combination tablets marketed by Gilead as TRUVADA®, which contains 300 mg TDF (tenofovir disoproxil fumarate) and 200 mg FTC (emtricitabine, EMTRIVA®), DESCOVY® 25 mg TAF (tenofovir alafenamide) and 200 mg FTC (emtricitabine). Tenofovir is available in five triple-drug combination tablets: ATRIPLA® (600 mg efavirenz, 200 mg FTC (emtricitabine), and 300 mg TDF (tenofovir disoproxil fumarate), EVIPLERA® (25 mg rilpivirine, 200 mg FTC (emtricitabine), and 245 mg tenofovir), COMPLERA® (200 mg FTC (emtricitabine), 25 mg rilpivirine, and 300 mg TDF (tenofovir disoproxil fumarate)), BIKTARVY® (50 mg bictegravir, 200 mg FTC (emtricitabine), and 25 mg TAF (tenofovir alafenamide)), ODEFSEY® (200 mg FTC (emtricitabine), 25 mg rilpivirine, and 25 mg TAF (tenofovir alafenamide)), all of which are also marketed by Gilead. Tenofovir is available in two four-drug combination tablets: STRIBILD® (150 mg elvitegravir, 150 mg cobicistat, 200 mg FTC (emtricitabine), and 300 mg TDF (tenofovir disoproxil fumarate)), GENVOYA® (150 mg elvitegravir, 150 mg cobicistat, 200 mg FTC (emtricitabine), and 10 mg TAF (tenofovir alafenamide)), both of which are also marketed by Gilead.

Pre-exposure prophylaxis (PrEP) with oral tenofovir disoproxil fumarate/emtricitabine (TDF/FTC) is one of the most effective strategies to prevent HIV acquisition among at-risk individuals (Grant et al., N Engl J Med., 363(27): 2587-2599 (2010); Thigpen et al., N Engl J Med., 367(5): 423-434 (2012), Choopanya et al., Lancet, 381(9883): 2083-2090 (2013); and Baeten et al., N Engl J Med., 367(5): 399-410 (2012)). More recently, oral tenofovir alafenamide (TAF) has been approved for PrEP and exhibits improved properties relative to TDF (Ray et al., Antiviral Research, 125: 63-70 (2016); and De Clercq, E., Biochemical Pharmacology, 119: 1-7 (2016)). PrEP is now broadly recommended by the Centers for Disease Control and Prevention (CDC) and the World Health Organization (WHO), and is entering a phase of global implementation (Centers for Disease Control (CDC). Preexposure Prophylaxis for the Prevention of HIV in the United States: A Clinical Practice Guideline —2017 Update (published April 2018); World Health Organization (WHO). Guideline on when to start antiretroviral therapy and on pre-exposure prophylaxis for HIV, Sep. 30, 2015).

The PrEP trials and studies to date, however, have highlighted three major considerations that should be addressed to increase PrEP effectiveness: (1) the relationship between adherence and effectiveness (Amico, K. R., Curr Opin HIV AIDS, 7(6): 542-548 (2012), (2) pharmacologic measures predict the efficacy of PrEP more accurately than self-reported adherence (Van Damme et al., N Engl J Med., 367(5): 411-422 (2012); Marrazzo et al., N Engl J Med., 372(6): 509-518 (2015); Agot et al., AIDS Behav., 19(5): 743-751 (2015); Blumenthal, J. and Haubrich, R., Expert Opin Pharmacother., 14(13):1777-1785 (2013); Corneli et al., J Acquir Immune Defic Syndr., 68(5): 578-584 (2015); van der Straten et al., J. Int AIDS Soc., 19(1): 20642 (2016)), and (3) real-time monitoring of PrEP drug levels (Gupta et al., Hypertension, 70(5): 1042-1048 (2017); and Checchi et al., JAMA, 312(12): 1237-1247 (2014)) may improve subsequent PrEP drug-taking. The compositions and methods described herein address these concerns.

The disclosure provides a polyclonal antibody composition comprising a heterogeneous population of mammalian antibodies that specifically bind tenofovir (TFV) or a tenofovir derivative. The structure of tenofovir is set forth below:

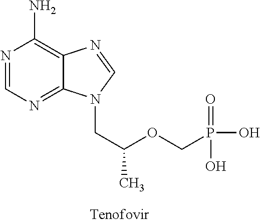 [SMILES: C[C@H](Cn1cnc2c(N)ncnc21)OCP(=O)(O)O]

Alternatively, the heterogeneous population of mammalian antibodies may specifically bind to a derivative of tenofovir. In certain embodiments, the heterogeneous population of mammalian antibodies is generated against an immunogen comprising a tenofovir derivative conjugated to a protein, which immunogen is a compound of formula (I):

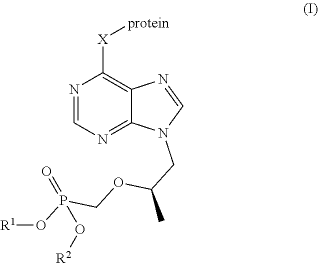

or a pharmaceutically acceptable salt thereof, wherein: R¹ and R² are each independently selected from the group consisting of hydrogen, C₁-C₆ alkyl, C₂-C₆ alkenyl, C₂-C₆ alkynyl, aryl, arylalkyl, cyanoalkyl, and —(C₁-C₆-alkylene)-Y—(C₁-C₆ alkyl), wherein Y is selected from —O—, —NH—, —S—, —C(O)NH—, —C(O)O—, —C(O)S—, —OC(O)NH—, —OC(O)O—, and —NHC(O)NH—; and X is a linker.

In some embodiments, R¹ and R² are each hydrogen. In some embodiments, R¹ and R² are each —(C₁-C₆-alkylene)-Y—(C₁-C₆ alkyl), wherein Y is —OC(O)O—. In some embodiments, R¹ and R² are each —CH₂OC(O)OCH(CH₃)₂.

The group X is a linker moiety that links the protein to the remainder of the compound of formula (I). In some embodiments, X comprises a moiety derived from the reaction of two reactive groups, such as reactive groups R^{A} and R^{B}, wherein reaction between the groups R^{A} and R^{B} results in a moiety that covalently links the protein to the remainder of the compound of formula (I). For example, the group R^{A} may be a reactive group present on an amino acid side chain on the protein, such as an amine (e.g., from a lysine residue), a thiol (e.g., from a cysteine residue), or a carboxylic acid (e.g., from an aspartate or glutamate residue). The group R^{B} may be a reactive group that reacts with the amino acid side chain, such as an isothiocyanate, an isocyanate, a primary amine, a maleimide, a succinimidyl ester, a haloacetyl group, or the like.

In some embodiments, X comprises a moiety selected from the group consisting of:

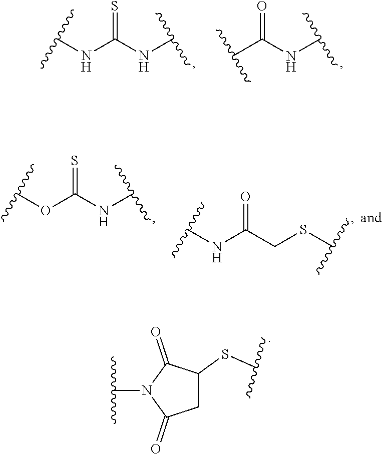 [SMILES: *NC(=S)N* ; *NC(*)=O ; *NC(=S)O* ; *NC(=O)CS* ; *SC1CC(=O)N(*)C1=O]

One skilled in the art will recognize that these moieties are derived from the reaction of two reactive groups such as those discussed above. For example, the thiourea is the reaction product of an isothiocyanate with a primary amine, the amide is the reaction product of a succinimidyl ester with a primary amine, etc.

In some embodiments, X may also include one or more additional groups of linking atoms, such as alkylene, heteroalkylene, arylene, heteroarylene, cycloalkylene, or heterocyclylene groups.

In some embodiments, X is:

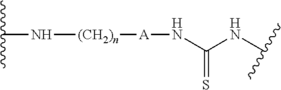

wherein: n is 1, 2, 3, or 4; and A is selected from aryl, heteroaryl, cycloalkyl, and heterocyclyl.

In some embodiments, X is:

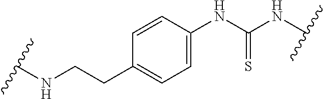 [SMILES: *NCCc1ccc(NC(=S)N*)cc1]

In some embodiments, the protein may be any protein of more than 2 kDa molecular weight, such as, for example, thyroglobulin, albumin, or hemocyanin.

In some embodiments, the immunogen is a compound of formula:

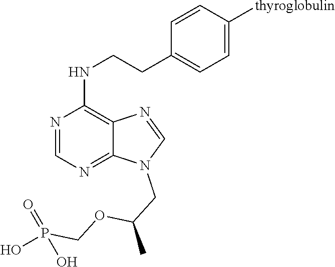

or a pharmaceutically acceptable salt thereof.

In some embodiments, the heterogeneous population of mammalian antibodies is generated against an immunogen comprising a tenofovir derivative compound of formula (II).

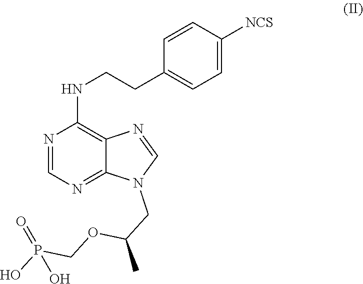

or a pharmaceutically acceptable salt thereof.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron, 33: 2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972).

The term “pharmaceutically acceptable salt” refers to a salt of a compound that is prepared with relatively nontoxic acids or bases, depending on the particular substituents found on the compounds described herein. When a compound of the present disclosure contains relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When a compound of the present disclosure contains relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science, 66: 1-19 (1977)). Certain specific compounds of the present disclosure may contain both basic and acidic functionalities that allow the compounds to be converted into either base or acid addition salts. These salts may be prepared by methods known to those skilled in the art.

Compounds disclosed herein may be synthesized, for example, according to the synthetic methods known in the art. The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in Vogel's Textbook ofPractical Organic Chemistry, 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Reactions can be worked up in the conventional manner, e.g. by eliminating the solvent from the residue and further purifying the desired compound according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in PGM Wuts and T W Greene, in Greene's book titled Protective Groups in Organic Synthesis (4^{th} ed.), John Wiley & Sons, New York (2006).

### Polyclonal Antibody Composition

The polyclonal antibody composition described herein may be produced by (a) administering to an animal the above-described immunogen; and (b) isolating from the animal an antibody that specifically binds to tenofovir or a tenofovir derivative. The immunogen may be administered to any suitable animal, such that the animal is “immunized” against the immunogen or antigen. Suitable animals for antibody production include, but are not limited to mice, rats, hamsters, guinea pigs, rabbits, cats, dogs, pigs, sheep, goats, horses, and cows. The animal desirably is a mouse, rat, hamster, guinea pig, or rabbit.

Polyclonal antibodies typically are produced by immunizing an animal with an immunogen (such as is described herein) in combination with an adjuvant, such as Freund's complete adjuvant, Freund's incomplete adjuvant, water-in-oil emulsions (e.g., Specol), and oil-in-water emulsions (e.g., RIBI Adjuvant System® (RAS), Sigma-Aldrich, St. Louis, Mo.) (see, e.g., Stils, Jr., H. F., ILAR Journal, 46(Issue 3): 280-293 (2005)). Immunization can be carried out using conventional methods, such as those described in, e.g., Schunk, M. K., and Macallum, G. E., ILAR Journal, 46(Issue 3): 241-257 (2005); G. C. Howard and D. R. Bethell (eds.), Basic Methods in Antibody Production and Characterization (Routledge Revivals), 1st Edition, CRC Press (2019); and Hanly et al., ILAR Journal, 37: 93-118 (1995)).

While the composition desirably comprises polyclonal antibodies, in some embodiments the composition may comprise a monoclonal antibody generated against a compound of formula (I) or formula (II) disclosed herein. Monoclonal antibodies typically are produced using hybridoma technology, as first described in Kohler and Milstein, Eur. J Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J. Mol. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)).

Following immunization, antibody titers in the animal can be monitored to determine the stage of immunization desired, which stage corresponds to the amount of enrichment or biasing of the repertoire desired. Partially immunized animals typically receive only one immunization and antibody-producing cells are collected therefrom shortly after a response is detected. Fully immunized animals display a peak titer, which is achieved with one or more repeated injections of the immunogen or antigen into the host mammal, typically at 2-3 week intervals.

Once the desired antibody titer is achieved in an immunized animal, the antibody of interest is isolated and purified from the animal. Antibody purification typically involves isolation of antibody from serum (for polyclonal antibodies) or from ascites fluid or culture supernatant of a hybridoma cell line (for monoclonal antibodies). Antibody purification methods are known in the art, and can be crude to highly specific. In this regard, crude purification methods involve precipitation of a subset of total serum proteins which includes antibodies. General antibody purification methods involve affinity purification of certain antibody classes (e.g., IgG) without regard to antigen specificity. In contrast, specific purification methods involve affinity purification of only those antibodies in a sample that bind to a particular antigen or immunogen. It will be appreciated that the degree to which the antibody is purified (crude, general, specific) depends on the intended application of the antibody.

The polyclonal antibodies produced by the above-described methods may be in the form of a composition which comprises a heterogeneous population of mammalian antibodies. The composition desirably is a pharmaceutically acceptable (e.g., physiologically acceptable) composition, which comprises a carrier, preferably a pharmaceutically acceptable (e.g., physiologically acceptable) carrier, and the heterologous population of mammalian antibodies (e.g., polyclonal antibodies). Any suitable carrier can be used within the context of the disclosure, and such carriers are well known in the art. For example, the composition may contain preservatives, such as, for example, methylparaben, propylparaben, sodium benzoate, and benzalkonium chloride. A mixture of two or more preservatives optionally may be used. In addition, buffering agents may be included in the composition. Suitable buffering agents include, for example, citric acid, sodium citrate, phosphoric acid, potassium phosphate, and various other acids and salts. A mixture of two or more buffering agents optionally may be used. Methods for preparing compositions for pharmaceutical use are known to those skilled in the art and are described in, for example, Remington: The Science and Practice of Pharmacy, Lippincott Williams & Wilkins; 21st ed. (May 1, 2005).

### Sample

The terms “sample,” “biological sample,” and “test sample” are used interchangeably herein and refer to a substance containing or suspected of containing tenofovir or a tenofovir derivative. The biological sample may be derived from any suitable source. In one embodiment, the source of the biological sample is a human bodily substance (e.g., blood, serum, plasma, urine, saliva, sweat, sputum, semen, mucus, lacrimal fluid, lymph fluid, amniotic fluid, interstitial fluid, lung lavage, cerebrospinal fluid, feces, hair, breast milk, tissue, an organ, etc.). In some embodiments, the sample is urine, serum, hair, or saliva. The sample may be a liquid sample, a liquid extract of a solid sample, a fluent particulate solid, or fluid suspension of solid particles.

The sample may be obtained from any suitable subject, but is ideally obtained from a human subject. In some embodiments, the subject is a human undergoing treatment with tenofovir or a derivative thereof. For example, the subject may be a human at risk for infection by human immunodeficiency virus (HIV), in which case the human may be undergoing Pre-Exposure Prophylaxis (“PrEP”) therapy and receiving a daily regimen of tenofovir and emtricitabine to prevent HIV infection, as discussed herein. Alternatively, the subject may be a human already infected with HIV or HBV, in which case the infected human may be receiving a daily dose of tenofovir alone, or in combination with other antiretroviral agents.

In some embodiments, a liquid biological sample may be diluted prior to use in an assay. For example, in embodiments where the sample is a human body fluid (e.g., serum, urine, or saliva), the fluid may be diluted with an appropriate solvent (e.g., PBS buffer). A fluid sample may be diluted about 1-fold, about 2-fold, about 3-fold, about 4-fold, about 5-fold, about 6-fold, about 10-fold, about 100-fold, or greater, prior to use.

In other embodiments, the sample may undergo pre-analytical processing. Pre-analytical processing may offer additional functionality, such as nonspecific protein removal and/or effective yet inexpensive implementable mixing functionality. General methods of pre-analytical processing include, for example, the use of electrokinetic trapping, AC electrokinetics, surface acoustic waves, isotachophoresis, dielectrophoresis, electrophoresis, and other pre-concentration techniques known in the art. In some cases, a liquid sample may be concentrated prior to use in an assay. For example, in embodiments where the sample is a human body fluid (e.g., serum, urine, or saliva), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

### Assay/Method

In order to detect tenofovir or a tenofovir in a sample, the present disclosure also provides a solid support which comprises the above-described polyclonal antibody composition immobilized thereon. The terms “solid phase” and “solid support” are used interchangeably herein and refer to any material that can be used to attach and/or attract and immobilize one or more antibodies. Any solid support known in the art can be used in the methods described herein. Examples of suitable solid supports include electrodes, test tubes, beads, microparticles, nanoparticles, wells of micro- or multi-well plates, gels, colloids, biological cells, sheets, strips (e.g., test strips), sample pads, and chips.

In one embodiment, a solid support desirably comprises a plurality (e.g., 2 or more, 50 or more, 100 or more, 1,000 or more, or 5,000 or more) of antibodies immobilized on the surface thereof which bind to tenofovir or a tenofovir derivative. The term “immobilized,” as used herein, refers to a stable association of a binding member with a surface of a solid support. Following a sufficient incubation time between the solid support and the sample, as discussed herein, tenofovir or a derivative thereof, if present in the sample, desirably is captured on the surface of the solid support via the immobilized antibody.

An antibody or antibody fragment may be attached to a solid support via a linkage, which may comprise any moiety, functionalization, or modification of the support and/or antibody that facilitates the attachment of the antibody to the support. The linkage between the antibody and the support may include one or more chemical or physical bonds (e.g., non-specific attachment via Van der Waals forces, hydrogen bonding, electrostatic interactions, hydrophobic/hydrophilic interactions, etc.) and/or chemical spacers providing such bond(s). Any number of techniques may be used to attach an antibody to a wide variety of solid supports (see, e.g., U.S. Pat. No. 5,620,850; and Heller, Acc. Chem. Res., 23: 128 (1990)).

The disclosure also provides a method of detecting tenofovir or a tenofovir derivative in a sample obtained from a subject, which method comprises (a) contacting a sample obtained from a subject with the solid support having the polyclonal antibody composition immobilized thereon, under conditions which allow binding of tenofovir or a tenofovir derivative, if present in the sample, to the polyclonal antibody composition, and (b) detecting binding of tenofovir or a tenofovir derivative bound to the polyclonal antibody composition.

Also provided is an assay for detecting the presence of tenofovir or a tenofovir derivative in a sample obtained from a subject, which comprises: (a) contacting a biological sample with the above-described polyclonal antibody composition, wherein the subject is undergoing treatment with tenofovir or a tenofovir derivative; and (b) detecting the polyclonal antibody composition bound to tenofovir or a tenofovir derivative. The terms “assay” and “biological assay,” as used herein, refer to a biological testing procedure for determining the presence or concentration of a substance or analyte in a sample, composition, or other bulk material.

In addition to being used to “capture” the tenofovir or tenofovir derivative in the sample, the polyclonal antibody composition may be used to detect binding of tenofovir bound to the polyclonal antibodies immobilized on the solid support. When the polyclonal antibody composition also is used for detection, at least a portion of the heterologous population of mammalian antibodies comprises a detectable label. In embodiments where the polyclonal antibody composition is not used as a “detection” antibody, binding of tenofovir or a tenofovir derivative to the immobilized polyclonal antibody composition results in the formation of a first complex, and the method further comprises contacting the sample with a conjugate comprising a second antibody and a detectable label attached thereto, wherein the conjugate binds to the first complex. In either case, the method further comprises assessing the presence of a signal from the detectable label, wherein the presence of a signal from the detectable label indicates the presence of tenofovir or a derivative thereof in the sample.

In some embodiments, the polyclonal antibody composition may be directly or indirectly labeled with a detectable label to facilitate detection of tenofovir (or derivatives thereof) bound to the polyclonal antibodies. Thus, in some embodiments, the method comprises (a) contacting a sample obtained from a subject with one or more polyclonal antibodies (such as a polyclonal antibody composition) which comprise a detectable label and specifically bind to tenofovir or a tenofovir derivative under conditions which allow binding of tenofovir or a derivative thereof, if present in the sample, to the polyclonal antibodies, and (b) assessing the presence of a signal from the detectable label, wherein the presence of a signal from the detectable label indicates the presence of tenofovir or a derivative thereof in the sample. In other embodiments, the method comprises (a) contacting a sample obtained from a subject with the polyclonal antibody composition under conditions which allow binding of tenofovir or a derivative thereof, if present in the sample, to the polyclonal antibody composition (also referred to as a “capture antibody”) to form a first complex; (b) contacting the sample with a conjugate comprising a second antibody (also referred to as a “detection antibody”) and a detectable label attached thereto, wherein the conjugate binds to the first complex; and (c) assessing the presence of a signal from the detectable label, wherein the presence of a signal from the detectable label indicates the presence of tenofovir or a derivative thereof in the sample.

The term “conjugate,” as used herein, refers to a complex comprising an antibody or antigen-binding fragment thereof and a detectable label. In the context of the present disclosure, the second antibody, or antigen-binding fragment thereof, portion of the conjugate specifically binds to a target antigen (e.g., tenofovir or a derivative thereof), which results in the linkage of the conjugate to the captured analyte and formation of an immunosandwich (also referred to herein as an “immunosandwich complex”). It will be appreciated that, in sandwich immunoassay formats, the first (capture) antibody and the second (detection) antibody recognize two different non-overlapping epitopes on a target analyte/antigen.

As discussed above, suitable detectable labels include various enzymes, prosthetic groups, fluorescent materials, luminescent materials, and radioactive materials (see, e.g., Zola, Monoclonal Antibodies: A Manual of Techniques, CRC Press, Inc. (1987)). For example, the detectable label can be a radioisotope (e.g., ³H, ¹⁴C, ³²P, ³⁵S, or ¹²⁵I), a fluorescent or chemiluminescent compound (e.g., fluorescein isothiocyanate, rhodamine, or luciferin), or an enzyme (e.g., alkaline phosphatase, beta-galactosidase, or horseradish peroxidase). Any method known in the art for separately conjugating an antibody to a detectable label may be employed in the context of the disclosure (see, e.g., Hunter et al., Nature, 144: 945 (1962); David et al., Biochemistry, 13: 1014 (1974); Pain et al., J. Immunol. Meth., 40: 219 (1981); and Nygren, J. Histochem. and Cytochem., 30: 407 (1982)). Signal generated from a detectable label attached to an antibody can be measured based on its spectroscopic properties.

The sample or solid support may be contacted with the polyclonal antibody composition using any suitable method known in the art. The term “contacting,” as used herein, refers to any type of combining action which brings an antibody, particular an antibody immobilized on a solid support, into sufficiently close proximity with an analyte of interest in a sample (e.g., tenofovir) such that a binding interaction will occur if the analyte of interest specific for the antibody is present in the sample. Contacting may be achieved in a variety of different ways, including directly combining the sample with the polyclonal antibody composition, or exposing the sample to a solid support comprising the polyclonal antibody composition immobilized thereon by introducing the solid support in close proximity to the sample. The contacting may be repeated as many times as necessary.

In some embodiments, the binding affinity between tenofovir, or a derivative thereof, and a polyclonal antibody should be sufficient to remain bound under the conditions of the assay, including wash steps to remove molecules or particles that are non-specifically bound. For example, the binding constant of the tenofovir or tenofovir derivative to a complementary antibody may be between at least about 10⁴ and about 10⁶ M⁻¹, at least about 10⁵ and about 10⁹ M⁻¹, at least about 10⁷ and about 10⁹ M⁻¹, greater than about 10⁹ M⁻¹, or greater. Contact between a solid support and the sample volume desirably is maintained (i.e., incubated) for a sufficient period of time to allow for the binding interaction between the tenofovir, or a derivative thereof, and an antibody to occur. In one embodiment, the sample volume is incubated with a solid support for at least 30 seconds and at most 10 minutes. For example, the sample may be incubated with the solid support for about 1, 2, 3, 4, 5, 6, 7, 8, or 9 minutes. In one embodiment, the sample may be incubated with the solid support for about 2 minutes. In addition, the incubating may be in a binding buffer that facilitates the specific binding interaction, such as, for example, albumin (e.g., BSA), non-ionic detergents (Tween-20, Triton X-100), and/or protease inhibitors (e.g., PMSF). The binding affinity and/or specificity of an antibody or antibody fragment may be manipulated or altered in the assay by varying the binding buffer. In some embodiments, the binding affinity and/or specificity may be increased by varying the binding buffer. In other embodiments, the binding affinity and/or specificity may be decreased by varying the binding buffer. Other conditions for the binding interaction, such as, for example, temperature and salt concentration, may also be determined empirically or may be based on manufacturer's instructions. For example, the contacting may be carried out at room temperature (21° C.-28° C., e.g., 23° C.-25° C.), 37° C., or 4° C.

Detecting binding of tenofovir, or a derivative thereof, to the polyclonal antibody composition desirably comprises the use of an immunoassay. The term “immunoassay,” as used herein, refers to a biochemical test that measures the presence or concentration of a macromolecule or a small molecule in a solution through the use of an antibody or an antigen. Any suitable immunoassay may be used, and a wide variety of immunoassay types, configurations, and formats are known in the art and within the scope of the present disclosure. Suitable types of immunoassays include, but are not limited to, enzyme-linked immunosorbent assay (ELISA), lateral flow assay (LFA) (also referred to as a “lateral flow immunoassay”), competitive inhibition immunoassay (e.g., forward and reverse), radioimmunoassay (RIA), fluoroimmunoassay (FIA), chemiluminescent immunoassay (CLIA), counting immunoassay (CIA), enzyme multiplied immunoassay technique (EMIT), one-step antibody detection assay, homogeneous assay, heterogeneous assay, capture on the fly assay, single molecule detection assay, etc. Such methods are disclosed in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792; International Patent Application Publications WO 2016/161402 and WO 2016/161400; and Adamczyk et al., Anal. Chim. Acta, 579(1): 61-67 (2006). In one embodiment, a lateral flow assay is used. Lateral flow assay (LFA) is a paper-based platform for the detection and quantification of analytes in complex mixtures, wherein the sample is placed on a test device and the results are displayed within 5-30 minutes (see, e.g., K. M. Koczula and A. Gallotta, Essays in Biochemistry, 60: 111-120 (2016)).

The immunoassay format may be “direct,” “indirect,” “sandwich,” or “competitive.” In a direct format, an antigen is directly adsorbed (immobilized) on the surface solid support (e.g., ELISA plate). The antigen is then detected by an antibody that is conjugated to an enzyme (e.g., horseradish peroxidase (HRP)). With indirect formats, antigen also is directly adsorbed onto the surface of a solid support, but a two-step detection process is employed: (1) unlabeled primary antibody is bound to the specific antigen followed by (2) application of an enzyme-conjugated secondary antibody directed against the host species of the primary antibody. Sandwich formats involve the use of capture and detection antigens to immobilize and detect an antigen in a sample. Specifically, the surface of a solid support is coated with a capture antibody, which capture antibody binds to and immobilizes a target antigen present in a sample applied thereto. A detection antibody is then added. The detection antibody can be directly labeled with an antibody (“direct sandwich immunoassay”) to allow for detection and quantification of the antigen. Alternatively, if the detection antibody is unlabeled, a secondary enzyme-conjugated detection antibody is required (“indirect sandwich assay”). Competitive formats are commonly used when an antigen is small and has only one epitope or antibody binding site, and involve labeling purified antigen instead of the antibody. Unlabeled antigen from samples and the labeled antigen compete for binding to the capture antibody. A decrease in signal from the purified antigen indicates the presence of the antigen in samples when compared to assay wells with labeled antigen alone.

Following reaction of the captured antigen (i.e., tenofovir or a derivative thereof) with a detectably labeled antibody or conjugate, any antibody, antibody fragment, or component of the conjugate not bound to the captured antigen may be removed, followed by an optional wash step. Any unbound antibody, antibody fragment, or component of the conjugate may be separated from an immunosandwich by any suitable means such as, for example, droplet actuation, electrophoresis, electrowetting, dielectrophoresis, electrostatic actuation, electric field mediated, electrode mediated, capillary force, chromatography, centrifugation, aspiration, or surface acoustic wave (SAW)-based washing methods.

It will be appreciated that different conformations of the antigen capture and immunosandwich formation methods described above are within the scope of the present disclosure. Indeed, the various components of the solid support, conjugates, and detectable labels described above may be arranged or utilized in any suitable combination, conformation, or format. For example, the disclosed methods may be performed in one-step, delayed one-step, or two-step format. Assay reagents (e.g., microparticles, conjugates, fluorophores, etc.) may be pre-mixed or added sequentially as appropriate.

The disclosed methods may comprise quality control components. “Quality control components” in the context of immunoassays and kits described herein, include, but are not limited to, calibrators, controls, and sensitivity panels. A “calibrator” or “standard” can be used (e.g., one or more, such as a plurality) in order to establish calibration (standard) curves for interpolation of the concentration of an analyte, such as an antigen. Alternatively, a single calibrator, which is near a reference level or control level (e.g., “low”, “medium”, or “high” levels), can be used. Multiple calibrators (i.e., more than one calibrator or a varying amount of calibrator(s)) can be used in conjunction to comprise a “sensitivity panel.” The calibrator is optionally part of a series of calibrators in which each of the calibrators differs from the other calibrators in the series, such as, for example, by concentration or detection method (e.g., colorimetric or fluorescent detection).

In certain embodiments, the methods described herein involve comparing the levels of tenofovir or a tenofovir derivative in a sample with a predetermined value or cutoff. The terms “predetermined cutoff,” “cutoff,” “predetermined value,” “reference level,” and “threshold level,” as used herein, refer to an assay cutoff value that is used to assess adherence to a tenofovir treatment regimen by comparing the assay results against the predetermined cutoff/level, where the predetermined cutoff/value already has been linked or associated with various clinical parameters (e.g., adherence to therapeutic regimen, presence of disease, stage of disease, severity of disease, progression, non-progression, improvement of disease, etc.). Cutoff values also may be used to assess diagnostic, prognostic, or therapeutic efficacy. It is well-known that cutoff values may vary depending on the nature of the detection method or assay. Whereas the precise value of the predetermined cutoff/value may vary between assays, the correlations as described herein should be generally applicable. Appropriate cutoff or threshold values can be determined or selected by those of ordinary skill in the art using routine methods. In some embodiments, an algorithm may be used to determine a predetermined value or threshold for decision making. Such an algorithm may consider a variety of factors, including, for example, (i) the age of the subject (e.g., higher threshold at higher age), (ii) HIV status, (iii) gender, and (iv) the sample (e.g., urine or serum).

The methods disclosed herein allow for detection of tenofovir or derivatives thereof at clinically relevant cutoff values of at least about 1,500 ng/mL in urine (e.g., about 1,600 ng/mL, 1,700 ng/mL, 1,800 ng/mL, 1,900 ng/mL, 2,000 ng/mL, 3,000 ng/mL, 4,000 ng/mL, 5,000 ng/mL or more) and at least about 10 ng/mL in serum (e.g., about 15 ng/mL, 20 ng/mL, 25 ng/mL, 30 ng/mL, 40 ng/mL, 50 ng/mL, 75 ng/mL, 100 ng/mL, 500 ng/mL or more). It will be appreciated that, in order to monitor adherence/compliance to a particular tenofovir treatment regimen, the methods for detecting tenofovir or a derivative thereof disclosed herein may be repeated two or more times during treatment. The methods may be repeated any number of times necessary to ensure accurate assessment of adherence to tenofovir therapy (e.g., 2, 3, 4, 5, 6, 7, 8, 9, 10, or more times).

### Kits and Instrumentation

Also provided herein are kits for performing the above-described methods. Instructions included in the kit may be affixed to packaging material or may be included as a package insert. The instructions may be written or printed materials but are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), etc. As used herein, the term “instructions” may include the address of an internet site that provides the instructions.

The kit may include a cartridge that includes a microfluidics module. In some embodiments, the microfluidics module may be integrated in a cartridge. The cartridge may be disposable. The cartridge may include one or more reagents useful for practicing the methods disclosed above. The cartridge may include one or more containers holding the reagents, as one or more separate compositions, or, optionally, as admixture where the compatibility of the reagents will allow. The cartridge may also include other material(s) that may be desirable from a user standpoint, such as buffer(s), a diluent(s), a standard(s) (e.g., calibrators and controls), and/or any other material useful in sample processing, washing, or conducting any other step of the assay.

The kit may further comprise reference standards for quantifying tenofovir or a derivative thereof present in the sample. The reference standards may be employed to establish standard curves for interpolation and/or extrapolation of the tenofovir or tenofovir derivative concentrations. The kit may include reference standards that vary in terms of concentration level. For example, the kit may include one or more reference standards with either a high concentration level, a medium concentration level, or a low concentration level. In terms of ranges of concentrations for the reference standard, this can be optimized per the assay.

The kit may also include quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics and are useful indicators of the integrity of the kit reagents and the standardization of assays.

The kit may also optionally include other reagents required to conduct an assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit may additionally include one or more other controls. One or more of the components of the kit can be lyophilized, in which case the kit can further comprise reagents suitable for the reconstitution of the lyophilized components. One or more of the components may be in liquid form.

The various components of the kit optionally are provided in suitable containers as necessary. The kit further can include containers for holding or storing a sample (e.g., a container or cartridge for a urine, saliva, plasma, or serum sample, or appropriate container for storing, transporting or processing tissue so as to create a tissue aspirate). Where appropriate, the kit optionally can contain reaction vessels, mixing vessels, and other components that facilitate the preparation of reagents or the test sample. The kit can also include one or more sample collection/acquisition instruments for assisting with obtaining a test sample, such as various blood collection/transfer devices (e.g., microsampling devices, micro-needles, or other minimally invasive pain-free blood collection methods; blood collection tube(s); lancets; capillary blood collection tubes; other single fingertip-prick blood collection methods; buccal swabs, nasal/throat swabs; 16-gauge or other size needle, surgical knife or laser (e.g., particularly hand-held), syringes, sterile container, or canula, for obtaining, storing, or aspirating tissue samples).

The concepts, kits, and methods as described herein can be implemented on any system or instrument, including any manual, automated, or semi-automated system. Ideally, the methods are performed using an automated or semi-automated system. In certain embodiments, the assays, kits, and kit components described herein can be implemented on electrochemical or other hand-held or point-of-care assay systems, such as, for example, the Abbott Point of Care (I-STAT®, Abbott Laboratories) electrochemical assay system that performs sandwich assays. Immunosensors and their methods of manufacture and operation in single-use test devices are described in, for example, U.S. Pat. Nos. 5,063,081; 7,419,821; 7,682,833; and 7,723,099 and U.S. Patent Application Publication No. 2004/0018577.

The following examples further illustrate the invention but, of course, should not be construed as in any way limiting its scope.

### Example 1

This example describes the development of an ELISA assay to detect tenofovir (TFV) in urine samples using the antibody disclosed herein.

It was hypothesized that TFV concentrations in urine, measured via an immunoassay, would correlate to those in plasma, the gold standard for short-term PrEP adherence in clinical trials, and associated with protection from HIV. To test this hypothesis, TFV levels were measured in stored urine samples collected from a randomly sampled cohort of HIV-negative men and women from the active PrEP arms in the Partners PrEP Study using enzyme-linked immunosorbent assay (ELISA) (lower limit of quantification [LLOQ] of 1000 ng/mL). Date-matched plasma TFV concentrations were measured using liquid chromatography-tandem mass spectrometry (LC-MS/MS) with an LLOQ of 0.31 ng/mL. Using the same cohort and a purposive sample of all HIV seroconverters on PrEP, a case-cohort analysis was conducted to assess the association between recent urine TFV level ≥1500 ng/mL, a threshold previously shown to accurately predict recent PrEP dosing, and protection from HIV. Weighted Cox proportional hazard models were used and adjusted for age, sex, and sexual behavior.

To assess the association between urine TFV concentrations ≥1,500 ng/mL and protection from HIV acquisition, a nested case-control analysis was conducted. Case samples collected on the date of the first evidence of HIV infection (i.e., first positive test for HIV-1 RNA) were matched with control samples collected at the same study visit month. If the case's first evidence of HIV was observed between regular urine sample archiving, controls from the nearest archive visit were selected. Control samples were matched 35:1, the ratio where estimates began to stabilize, and randomly sampled from the risk set of participants who were HIV-negative at the case's date of HIV detection, including future seroconverters. Controls could be matched to multiple cases. Conditional logistic regression, adjusted for matched sets, estimated the odds ratio of HIV acquisition given a urine TFV concentration ≥1,500 ng/mL, which approximates a rate ratio (RR) given the time-matched risk set sampling approach. Adjusted models controlled for participant sex, age, and report of any condomless sex with their study partner in the prior month at enrollment. All models were replicated to also assess the association of plasma TFV>40 ng/mL with HIV protection. Case samples were too few to conduct adequately powered sex-based subgroup analyses.

Of 4,432 individuals randomized to use of TDF or TDF/FTC in the Partners PrEP Study, 292 were included in the nested cohort. Among these participants, 39% were female and the median age was 33 (interquartile range [IQR]=28-39). Participants in the cohort contributed 722 paired urine and plasma samples. Of 52 individuals who seroconverted to HIV while using PrEP in the study, 22 had urine samples available from the visit where HIV was first detected and were included as cases. An additional 69 seroconverter samples collected prior to HIV infection were included as possible controls. Among cases, 55% were female and the median age was 33 (IQR=27-39).

The median duration from collection to assay of plasma and urine samples was 20 months and 103 months, respectively. In the cohort, the median TFV concentration was 37,500 ng/mL (IQR=500-90,000 ng/mL) in urine via ELISA and 65.4 ng/mL (IQR=1.6-103.0 ng/mL) in plasma via LC-MS/MS. Spearman's rank correlation coefficient (p) for the two measures was 0.46 (p<0.001). Of 558 plasma samples with detectable TFV (≥LLOQ of 0.31 ng/mL), 486 had a paired urine sample with detectable TFV (≥LLOQ of 1,000 ng/mL) for a sensitivity of 87% (95% CI=84-90%). There were 164 plasma samples with undetectable TFV, of which 119 had a paired urine sample with undetectable TFV for a specificity of 73% (95% CI=65-79%). Of 468 individuals with plasma TFV>40 ng/mL, 420 had a paired urine sample with TFV≥1,500 ng/mL, for a sensitivity of 90% (95% CI=87-92%). Finally, 254 plasma samples had TFV levels ≤40 ng/mL, of which 146 had a paired urine sample with TFV<1,500 ng/mL for a specificity of 57% (95% CI=51-64%).

In total, 770 control samples from 280 individuals were matched to the 22 case samples in this case-control study. Among participants in both active PrEP study arms, urine TFV≥1500 ng/mL was associated with a 71% (95% CI=30-88%) reduction in HIV risk in the adjusted model (Table 1). By contrast, plasma TFV>40 ng/mL was associated with an 87% (95% CI=54-96%) reduction in HIV risk.

TABLE 1
Percent HIV risk reduction associated with urine TFV concentrations
>1500 ng/mL as measured by a novel immunoassay

| n (%) with urine TFV ≥1500 ng/mL | | | | Adjusted % | |
|---|---|---|---|---|---|
| Case samples: | | % HIV risk | | HIV risk | |
| First evidence | Control | reduction^{a} | | reduction^{a,b} | Adjusted |
| of HIV | samples | (95% CI) | p-value | (95% CI) | p-value^{b} |
| 8/22 (36%) | 527/770 (68%) | 73% (36 to 89%) | 0.003 | 71% (30 to 88%) | 0.006 |

TFV: tenofovir,
CI: confidence interval
Analyses include individuals assigned to TDF/FTC or TDF-only PrEP. Estimates were generated using conditional logistic regression.
^{a}% risk reduction calculated as follows: (1 − RR)*100
^{b}Adjusted for sex, age at enrollment, and report of any condomless sex with study partner in the month prior to enrollment

Thus, in a large completed PrEP trial, urine TFV levels measured using the novel immunoassay described above were predictive of protection from HIV. Detection of TFV in urine showed good sensitivity and specificity for detection of TFV in plasma measured via LC-MS/MS, an established metric of short-term PrEP adherence. The results of this example suggest that use of a real-time assay to assess TFV levels in urine could be a valuable addition to existing objective metrics for PrEP adherence.

### Example 2

This example describes the development of a point-of-care (POC) lateral flow immunoassay (LFA) to detect tenofovir (TFV) in urine samples using the antibody disclosed herein.

The objective of this analysis was to compare a novel POC test for PrEP to laboratory-based ELISA in diverse patient populations. Urine samples were analyzed using the ELISA and POC LFA test from two cohorts of tenofovir disoproxil fumarate (TDF)-based PrEP users: the Partners PrEP Study, which recruited heterosexual men and women, and the I-BrEATHe Study, which recruited transwomen using estrogen and transmen using testosterone hormone therapy. Sensitivity, specificity, and accuracy of the POC test were calculated and compared to laboratory-based ELISA at cutoffs of 1,500 ng/mL and 4,500 ng/mL.

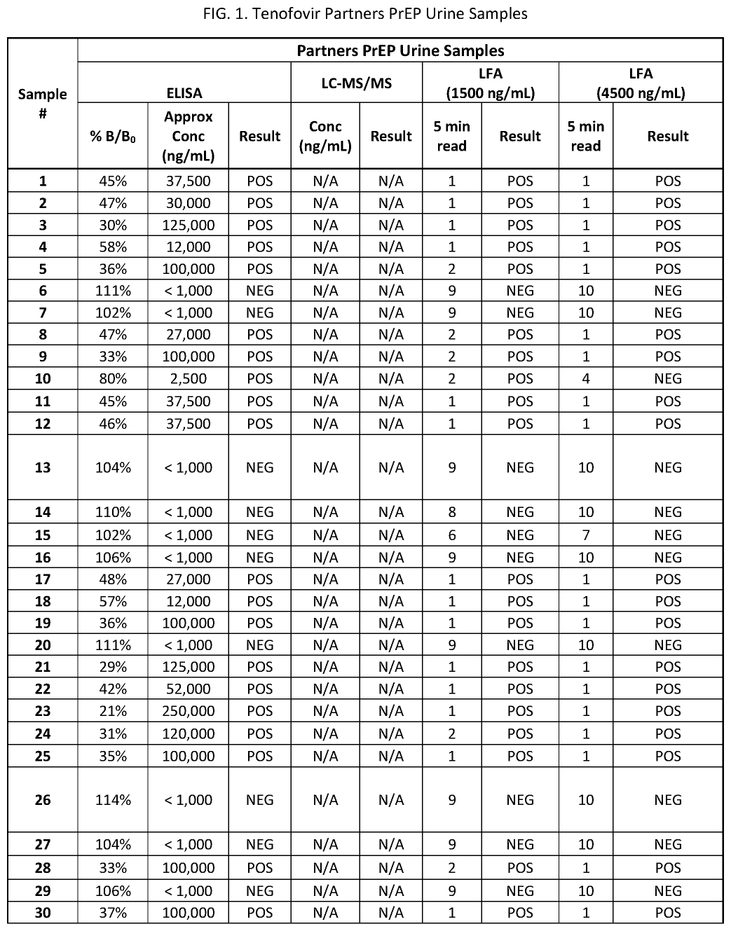

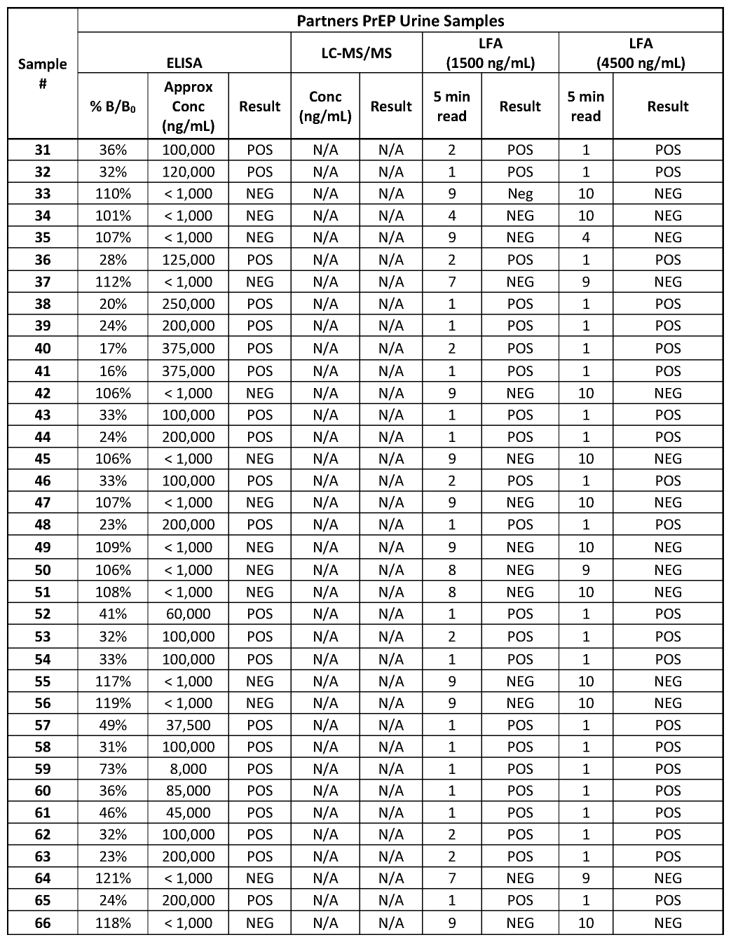

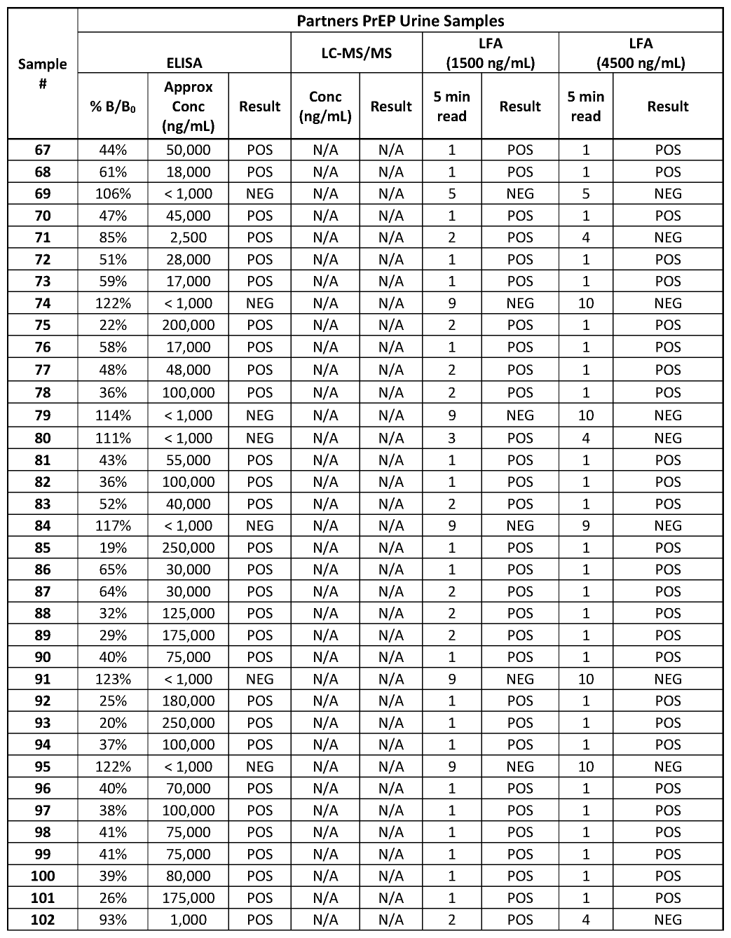

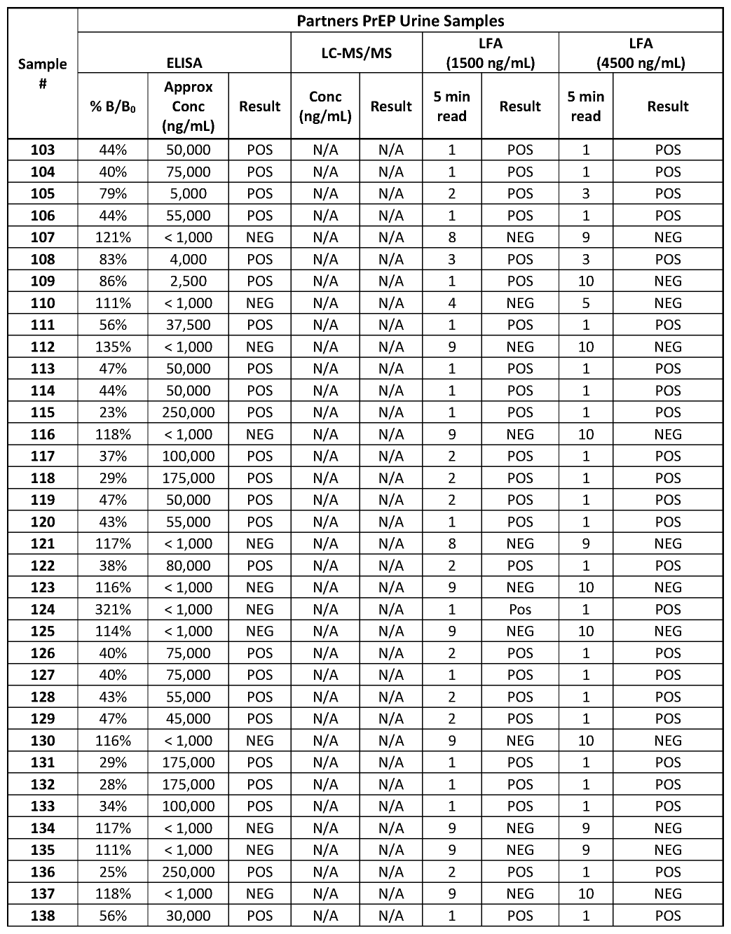

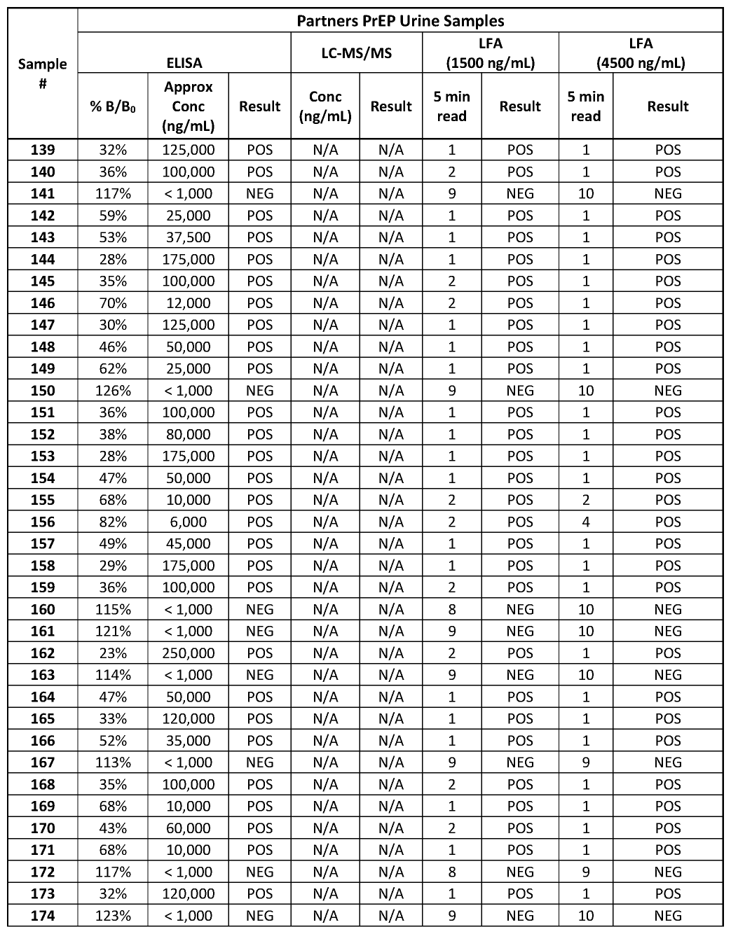

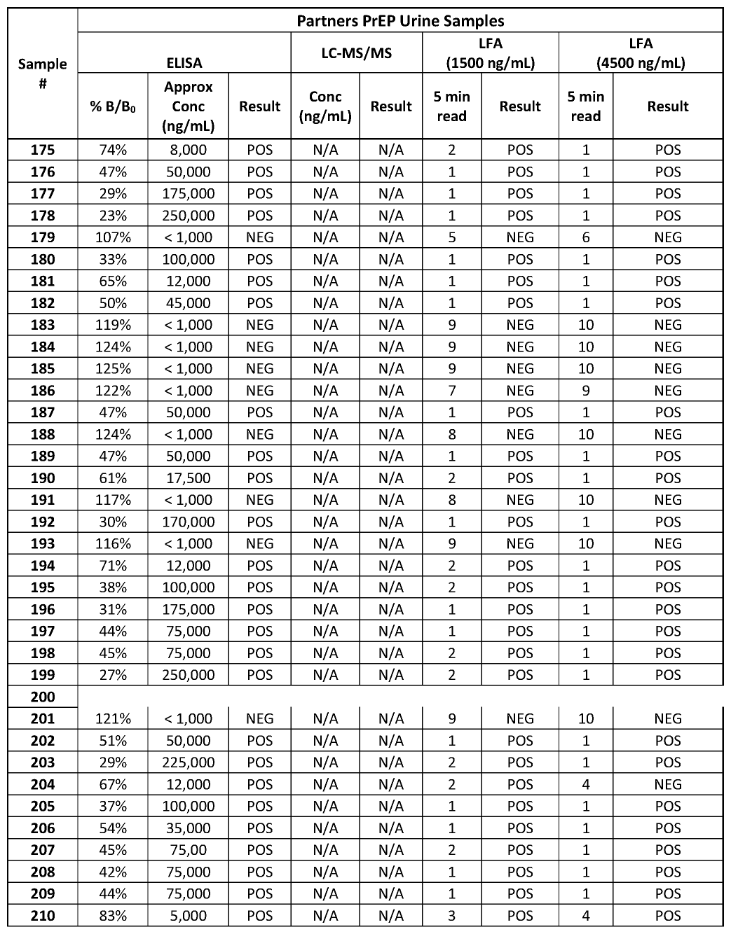

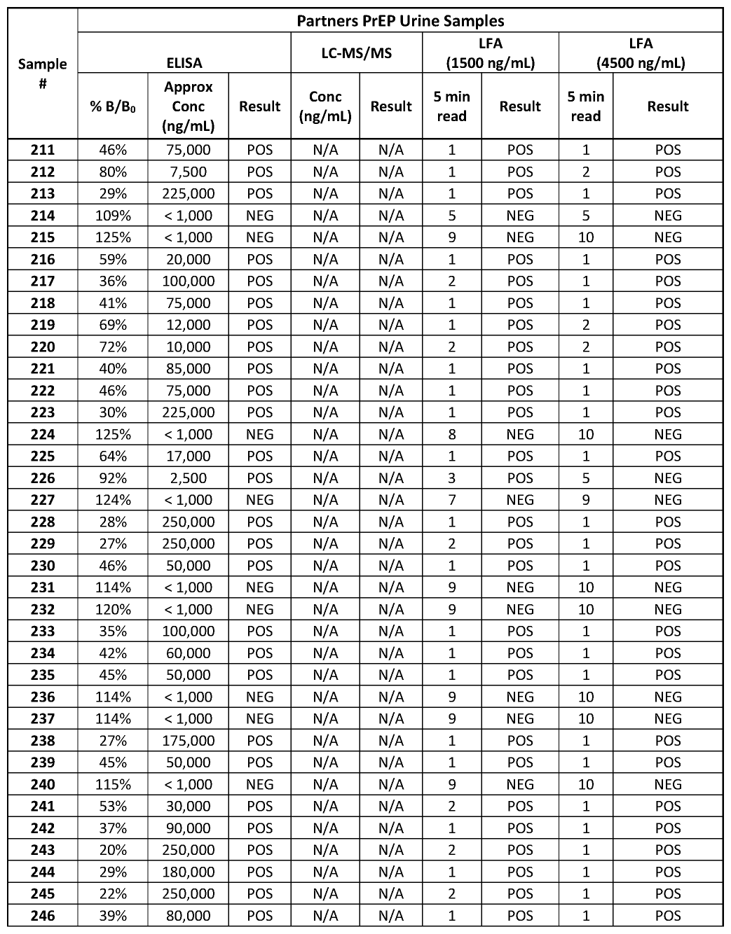

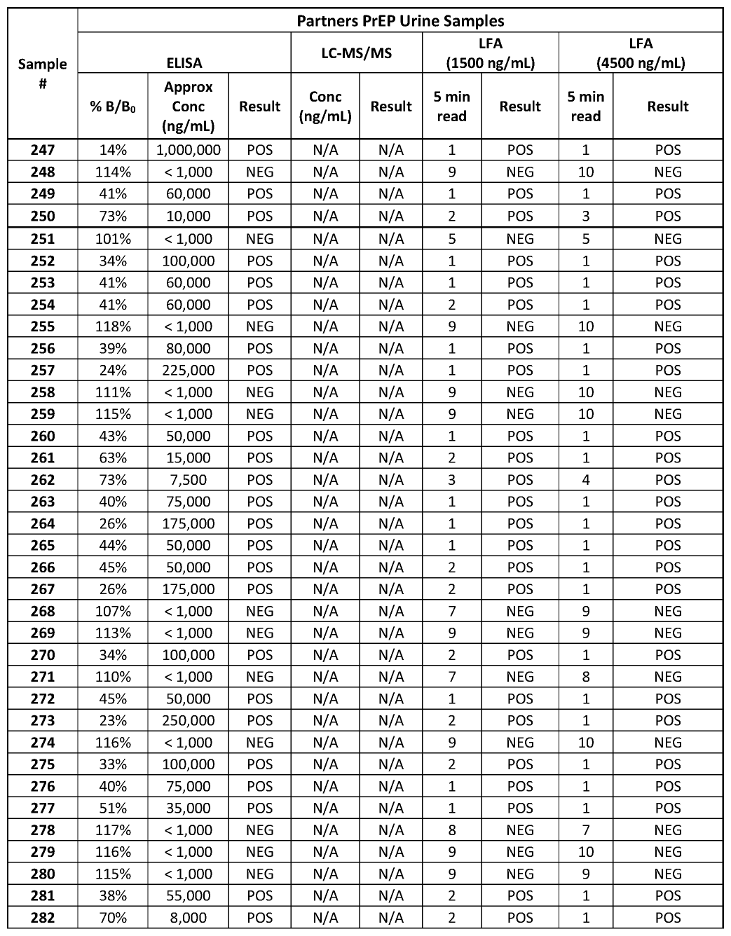

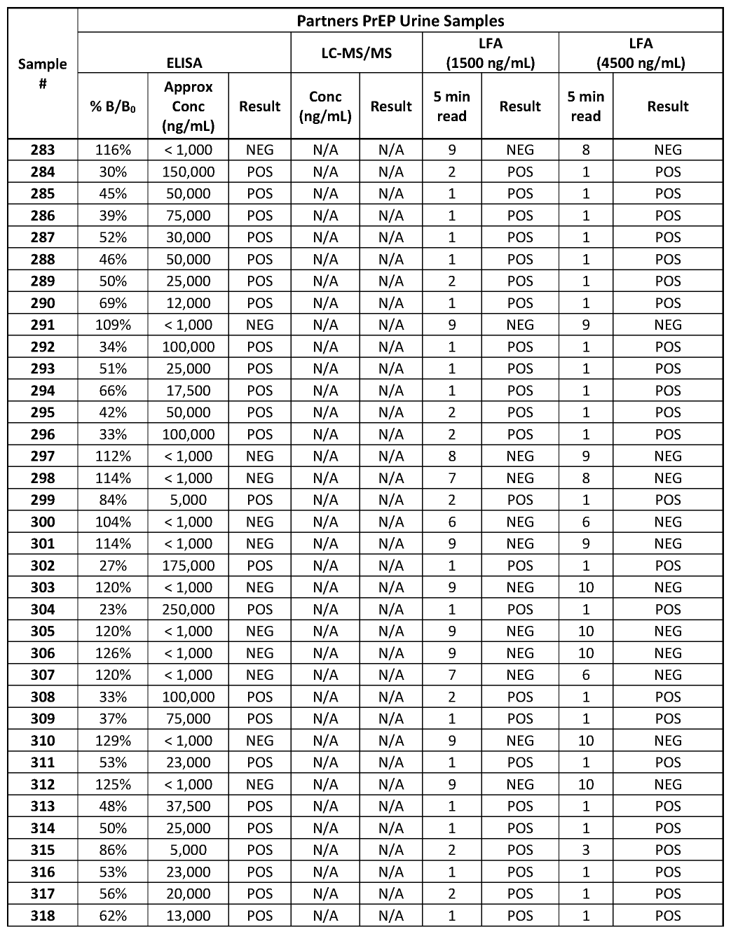

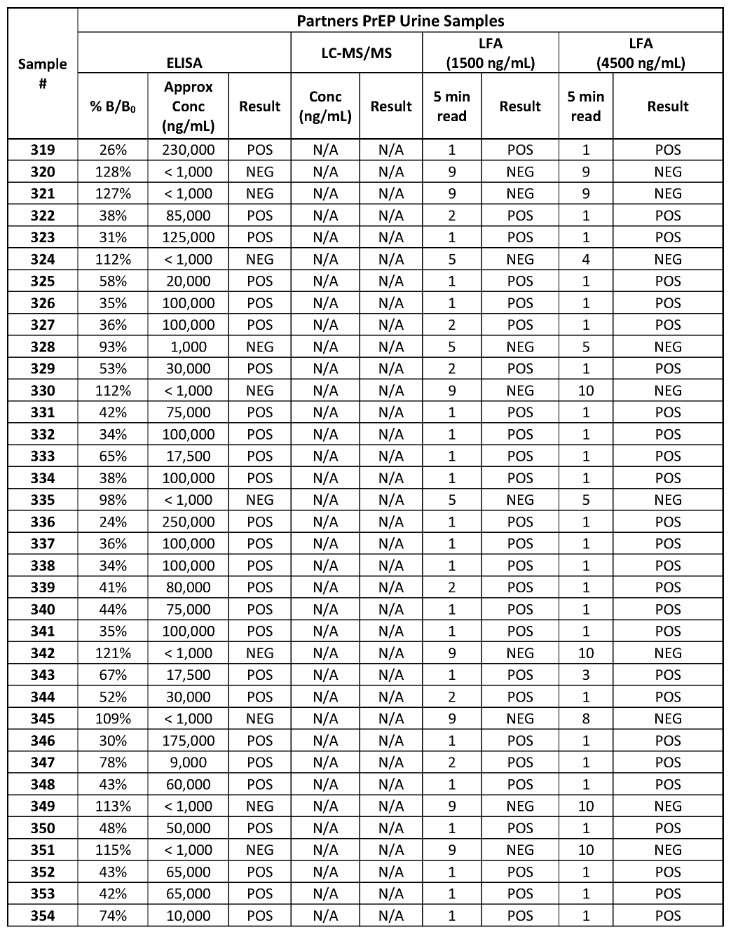

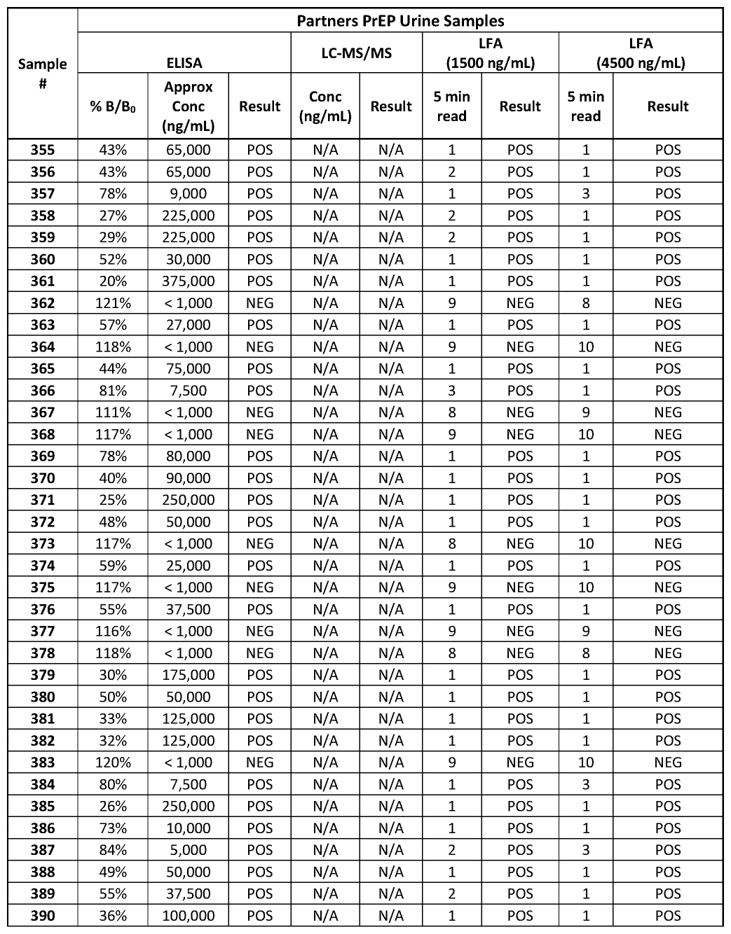

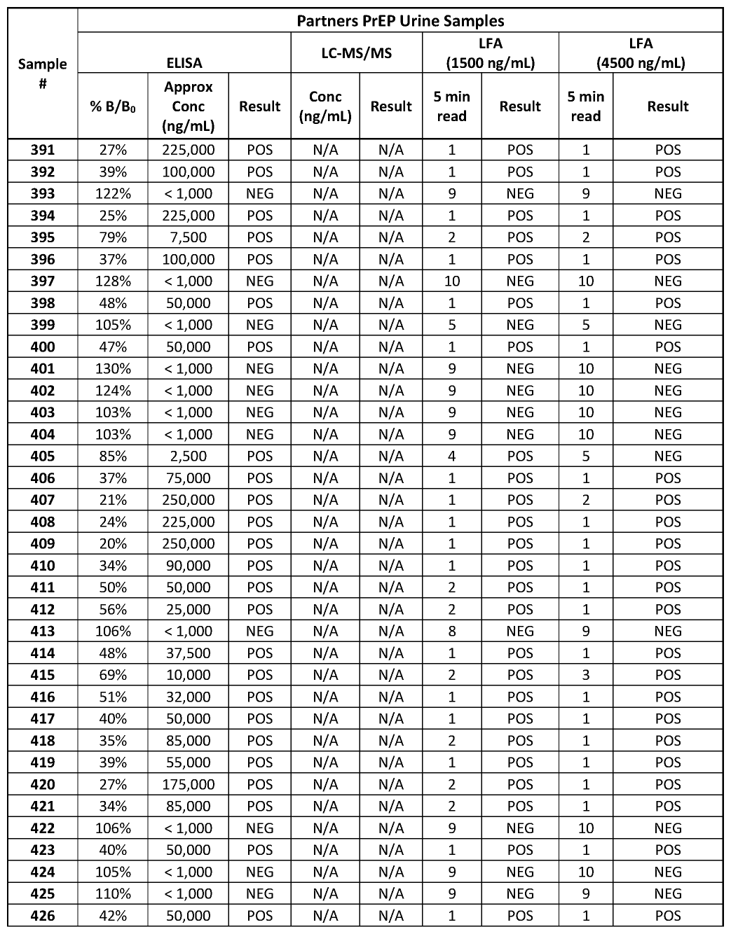

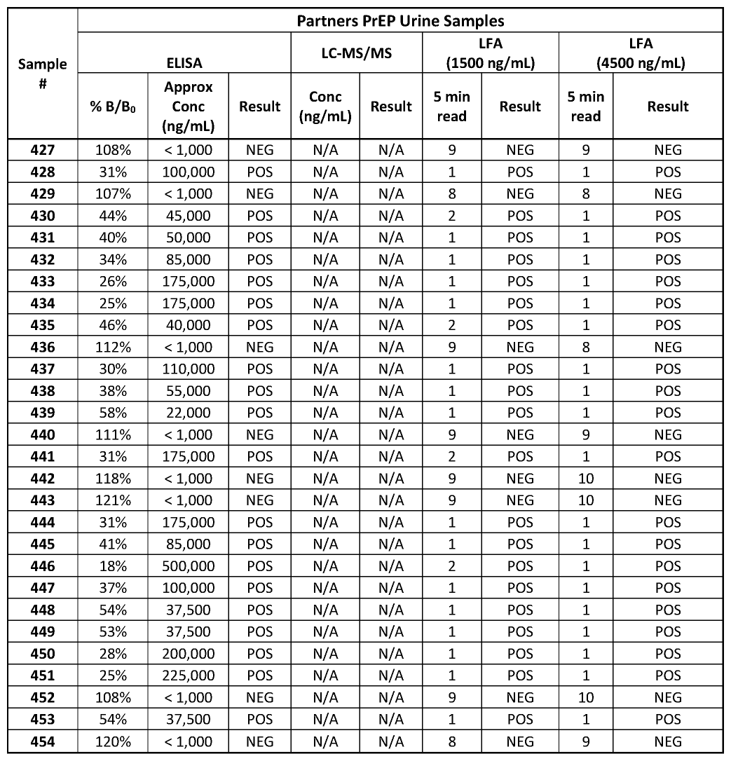

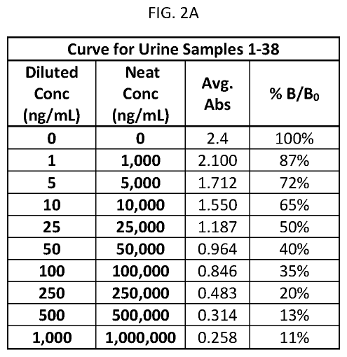

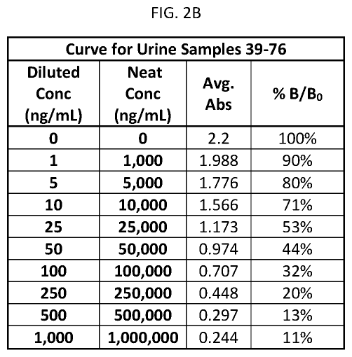

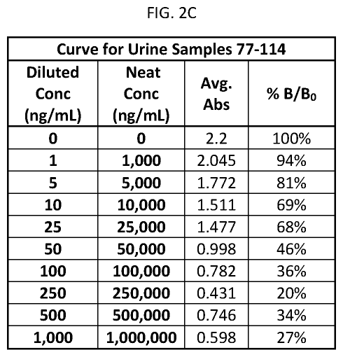

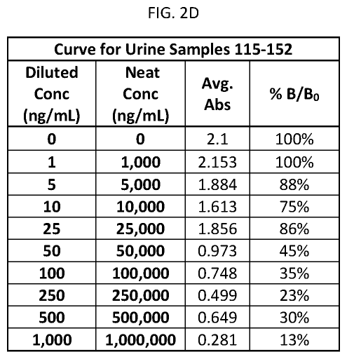

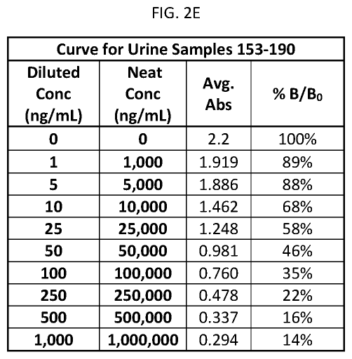

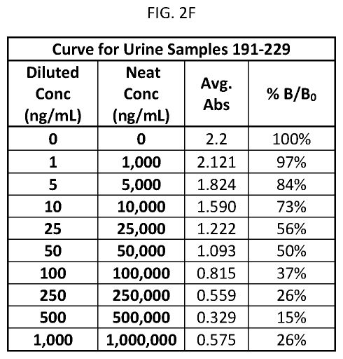

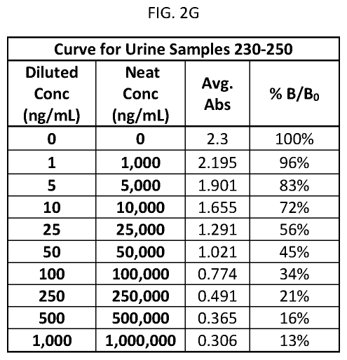

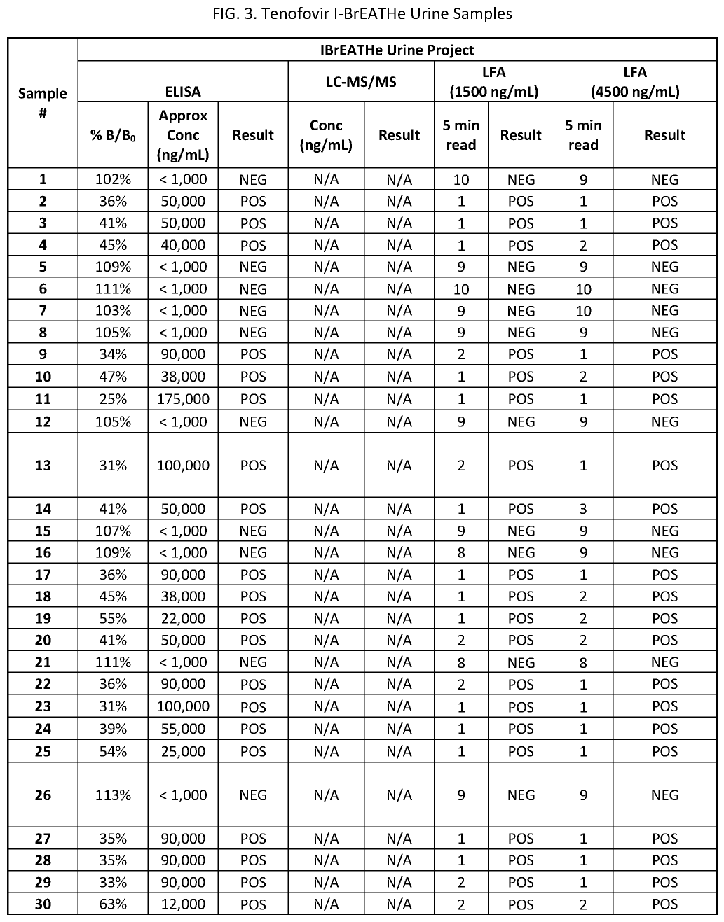

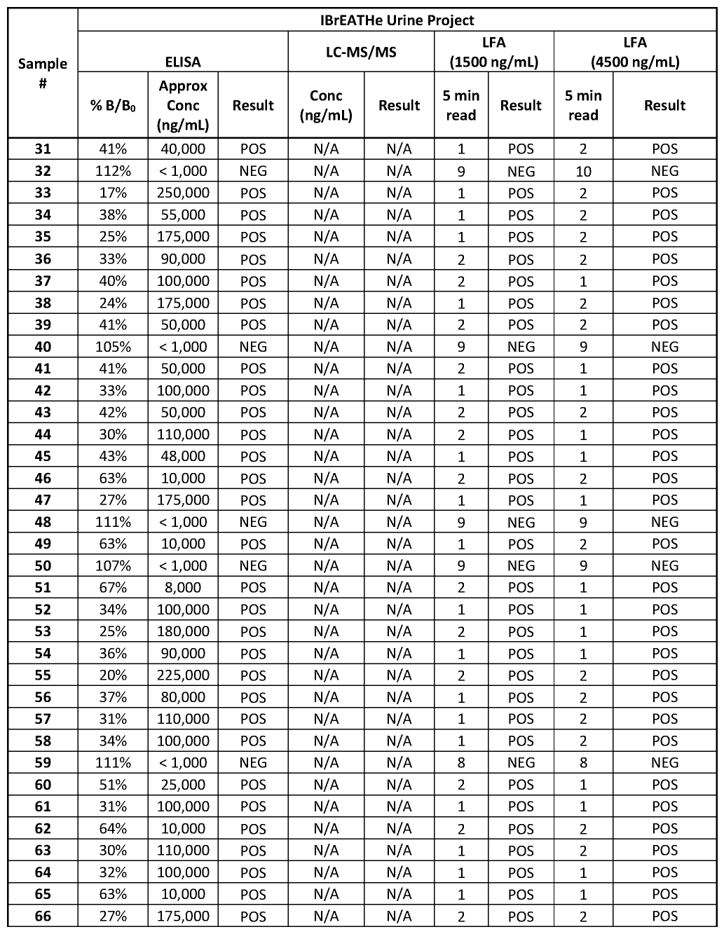

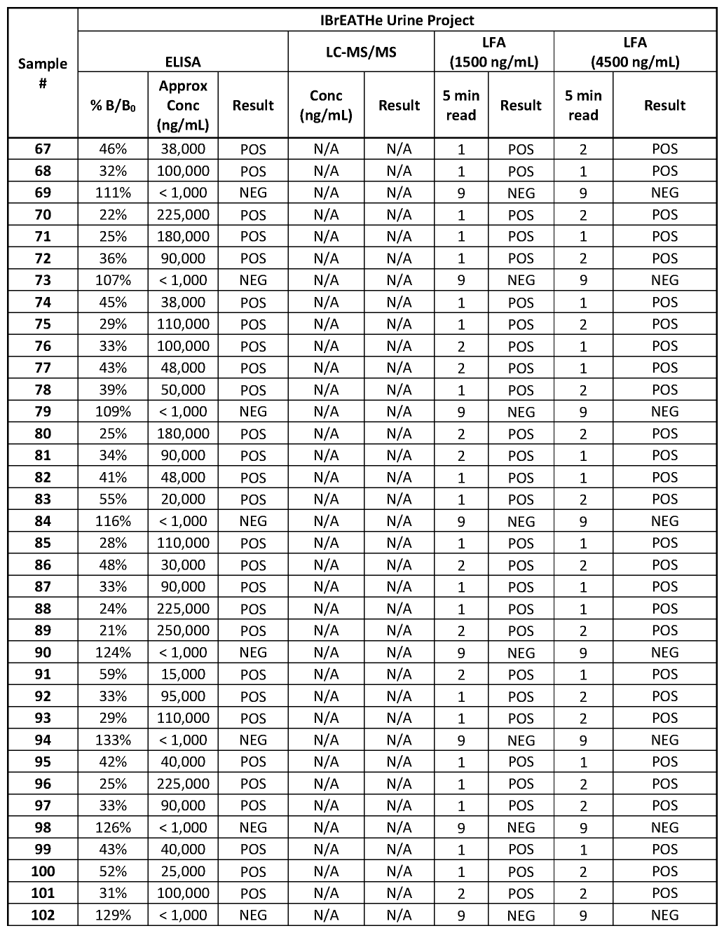

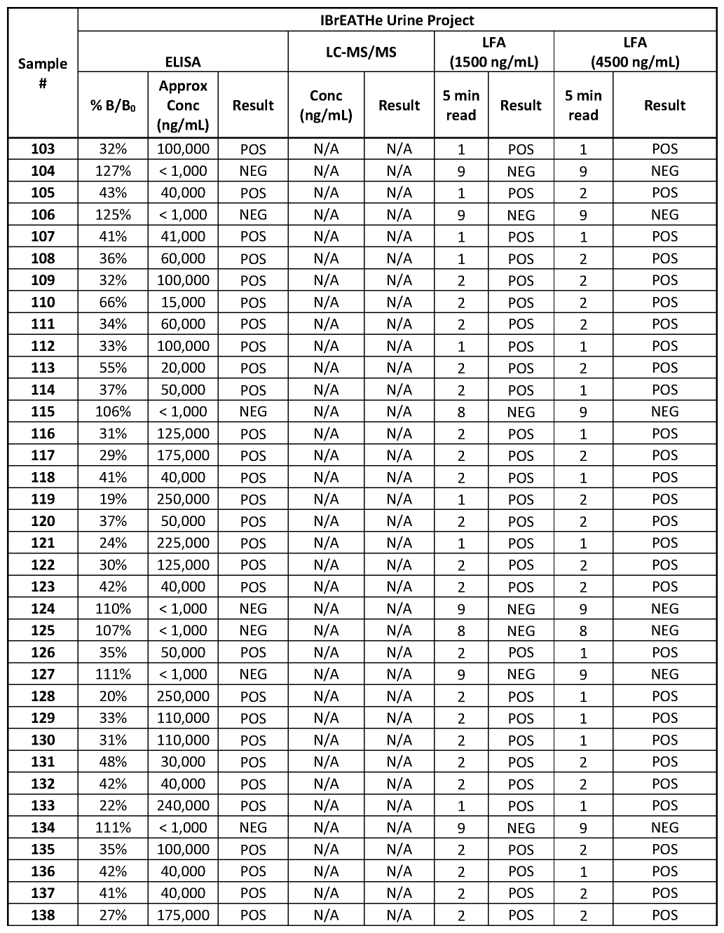

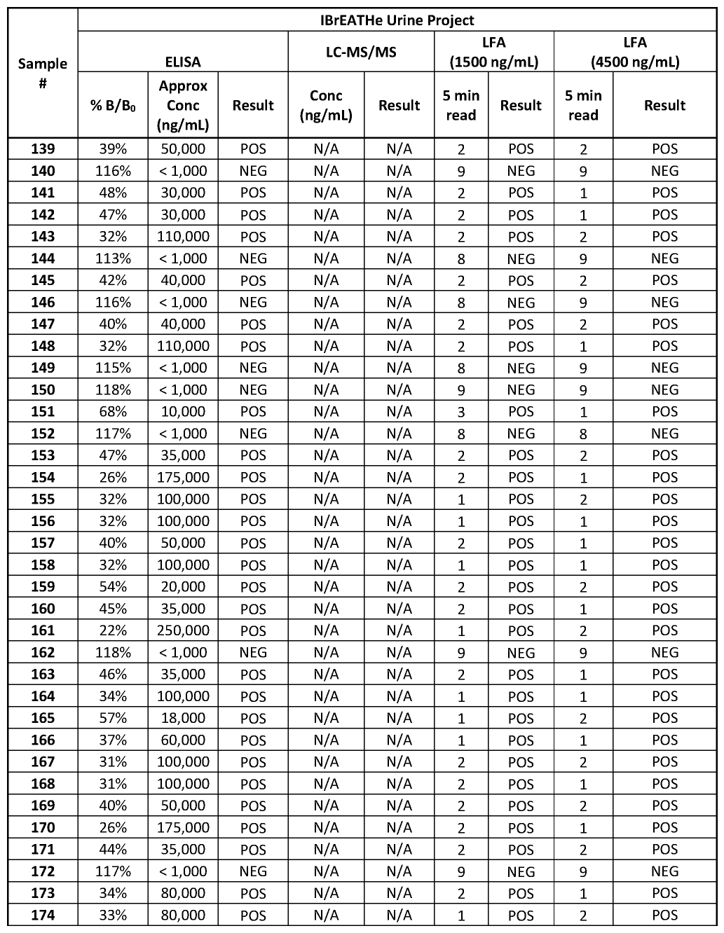

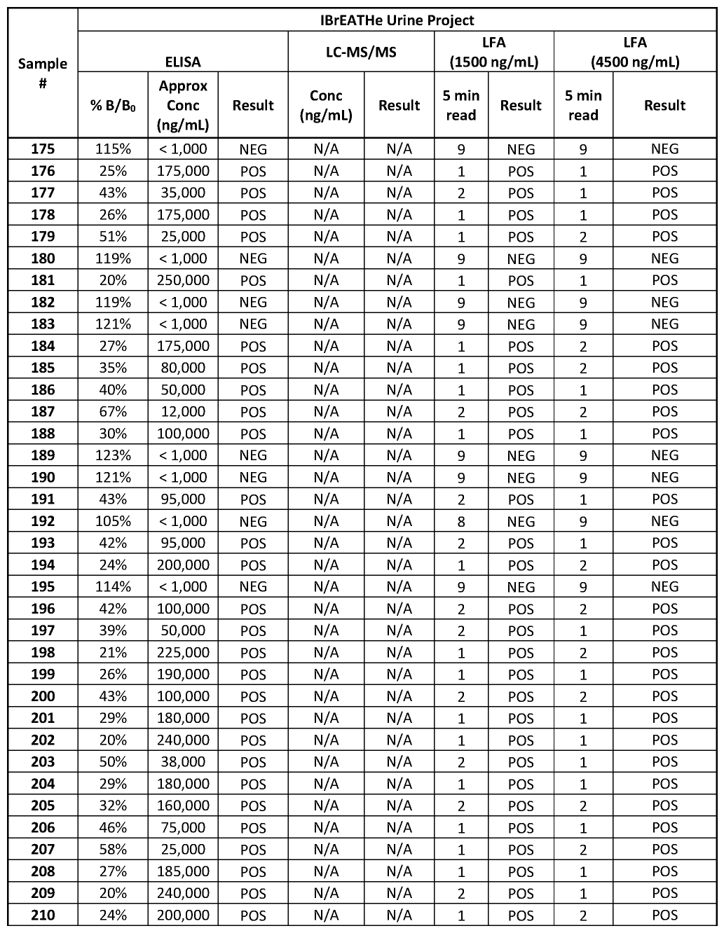

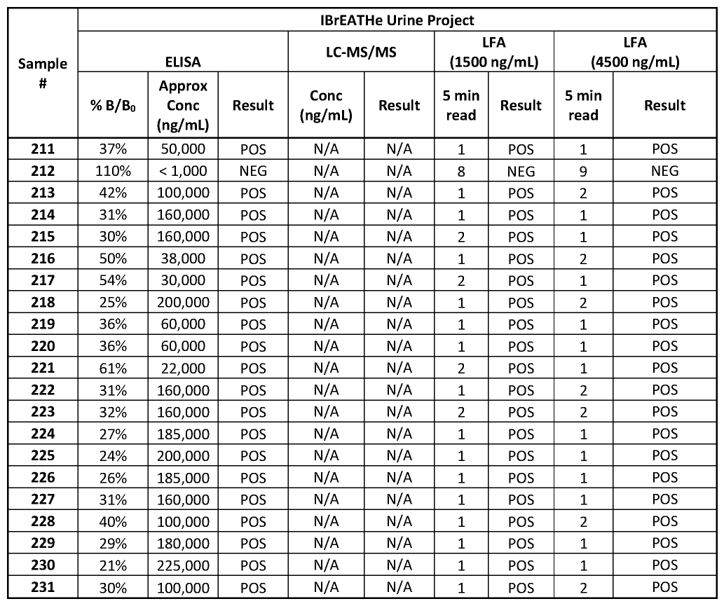

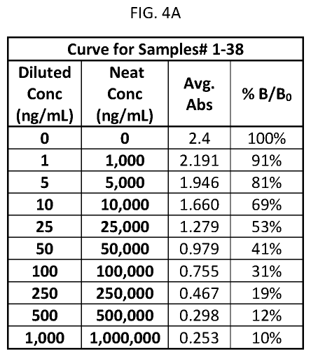

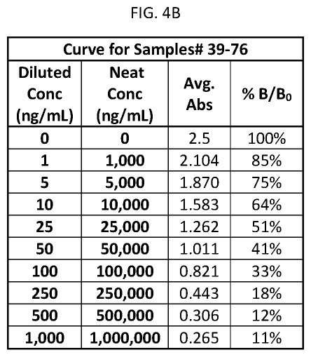

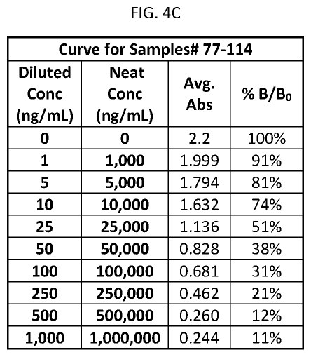

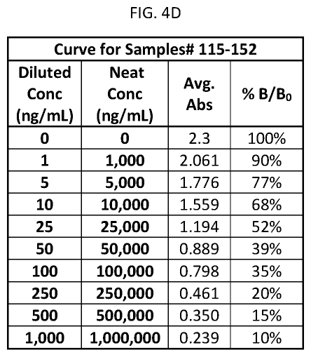

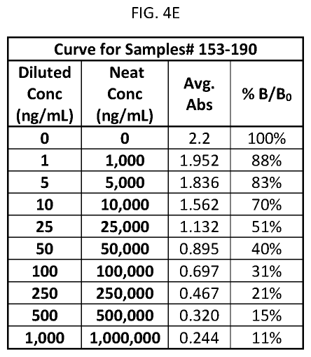

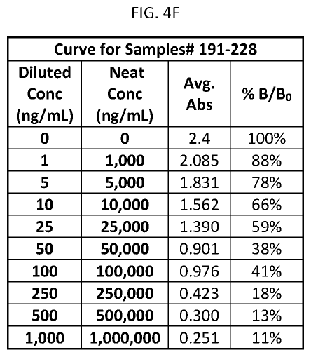

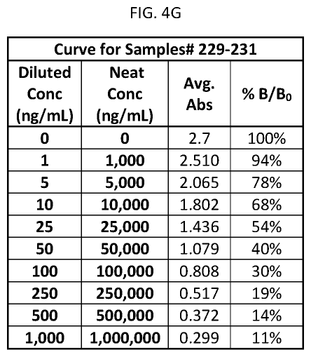

Overall, 684 urine samples were tested from 324 participants in the two cohorts. In Partners PrEP, 454 samples from 278 participants (41% women) were tested; the median age was 33 years (interquartile range (IQR) of 28-39). In I-BrEATHe, 231 samples from 46 individuals (50% transwomen) were tested; the median age was 31 years (IQR 25-40). Overall, of the 505 samples with TFV levels greater than or equal to the cutoff using laboratory-based ELISA, 505 of the POC test results were also positive, yielding 100% sensitivity. Of the 179 samples with TFV levels below the cut-off, 178 were negative with the POC test, yielding 99.4% specificity. The accuracy of the POC LFA was 99.8% compared to ELISA. Raw data comparing the results of LC-MS/MS, ELISA, and LFA assays are shown in FIG. 1-FIG. 4.

In 324 women and men (both cisgender and transgender) taking PrEP, the sensitivity, specificity, and accuracy of a novel POC test for urine TFV all exceeded 99% when compared to a laboratory-based ELISA method. Given the association of low urine TFV levels with HIV seroconversion events, the simplicity of using the LFA, and its expected low cost, this POC test is a promising tool to support adherence to PrEP that could be widely scalable to real-world clinical settings. The results of the example suggest the evaluation of adherence support using this point-of-care test in a randomized controlled trial.

### Example 3

This example describes a study to further validate a tenofovir immunoassay as described herein.

This study leveraged samples from TARGET, a directly observed therapy (DOT) randomized, open-label, clinical pharmacokinetic study of TDF/FTC in Thailand (Cressey et al., BMC Infect Dis., 17: 496 (2017). In TARGET, healthy participants were randomized (1:1:1) to 1 of 3 groups (10 participants each, total n=30) to receive directly observed doses of TDF 300 mg/FTC 200 mg for 6 weeks: Participants in group 1 received TDF/FTC once daily (“high adherence”); participants in group 2 received TDF/FTC 4 times/week (“moderate adherence”); and participants in group 3 received TDF/FTC 2 times/week (“low adherence”). Participants underwent direct observation of dosing Monday through Friday; drug ingestion on weekends was monitored by video/picture calls. Urine samples were collected and stored during 6 weeks of treatment administration and over 4 weeks of washout. The study was approved by Ethics Committees at the Institute for the Development of Human Research Protections at the Medical Sciences Department, Thai Ministry of Public Health; Sanpatong Hospital; and the University of Washington. The study was registered with ClinicalTrials.gov (#NCT0301260) and described in detail in Gandhi et al., J Acquir Immune Defic Syndr, 81: 72-77 (2019)).

Urine samples collected in TARGET were aliquoted for measurement by both liquid chromatography/tandem mass spectrometry (LC-MS/MS) and the immunoassay. Since TFV concentrates in urine (TRUVADA® (emtricitabine and tenofovir disoproxil fumarate) tablets package insert; Approved by U.S. Food and Drug Administration. 2004. Available at: gilead.com/;/media/files/pdfs/medicines/hiv/truvada/truvada_pi.pdf.; and Custodio et al., Antimicrob Agents Chemother., 60: 5135-5140 (2016)), and to compare TFV levels with those in the literature (Koenig et al., HIV Med., 18: 412-418 (2017)), urine samples were diluted 1:1000 before analysis. For the LC-MS/MS-based method, TFV was separated through reverse-phase high performance LC and quantified by MS/MS using electrospray positive ionization in multiple reaction monitoring mode [TFV, 287.9/175.9 (Q1/Q3)]. The lower limit of quantification (LLOQ) of the LC-MS/MS-based assay was 500 ng/mL. For the ELISA-based immunoassay, working solutions of TFV of known concentrations were prepared. Calibrators or different concentrations of TFV were incubated on a microtiter plate with the hapten to generate a dose-response curve. An ELISA plate reader extrapolated the concentration of TFV in the unknown specimen based on the calibration curve. The LLOQ for the ELISA-based immunoassay was 1,000 ng/mL.

To predict probabilities of being below different cutoffs of urine TFV levels for the POC assay, a mixed-effects interval regression model was used with log urine-immunoassay concentration as the dependent variable and days since the last dose as the independent variable. The analysis was restricted to spot urine samples obtained after 1 week of administration, to simulate urine collection at a clinic visit after TDF/FTC-based PrEP or ART has been started. Because food effects on TDF pharmacokinetics are minimal, food intake was not considered in the models. The probabilities of being below a given cutoff at any time since the last dose were calculated from the model using the estimated mean, person-to-person variation, and residual variation. Based on participant feedback from the previous studies that poor specificity tests were distressing (van der Straten et al., AIDS., 29: 2161-2171 (2015)); and van der Straten et al., A Qualitative Evaluation of Women's Experience Receiving Drug Feedback in MTN-025/HOPE—an HIV Prevention Open-Label Trial of the Dapivirine Vaginal ring. MTN-025/HOPE Study group. AIDS 2018 Conference. Amsterdam, The Netherlands; 2018, Abstract THPEC334. 2018. Available at: programme.aids2018.org). The focus was finding a cutoff with high specificity for dosing within 24 hours that still permitted adequate sensitivity for nonadherence. Because any dichotomization of time since last dose would gloss over some important distinctions, and because there were repeated measurements on the same individuals, a simple receiver operating characteristic curve was not examined.

Once an appropriate cutoff was determined, the sensitivity and specificity of the immunoassay was calculated compared with LC-MS/MS by cross-tabulating TFV levels above this cutoff versus below this cutoff by the two different assays. The Spearman correlation also was calculated between TFV levels generated by the two assays, using results from all urine samples in TARGET and then restricting the calculation to urine samples with detectable drug by both assays. Finally, agreement between urine TFV levels positive both by the immunoassay and LC-MS/MS was calculated using Bland-Altman methods (Bland J M, Altman D G, Lancet, 1: 307-310 (1986)).

Median TFV levels were 12,000 ng/mL by the immunoassay one day after dosing; 5,000 ng/mL 2 days after dosing; 1,500 ng/mL 3 days after dosing; and below the lower limit of quantification thereafter (≥4 days). An immunoassay cutoff of 1,500 ng/mL accurately classified 98% of patients who took a dose 24 hours prior as adherent. The specificity and sensitivity of the immunoassay compared with LC-MS/MS at the 1,500 ng/mL cutoff were 99% and 94%; the correlation between TFV levels by the two assays was high (0.92, P, 0.00001).

The results of this example demonstrate that a TFV immunoassay as described herein is highly specific, sensitive, and correlates strongly with LC-MS/MS measurements in a large DOT study.

### Example 4

This example describes the development of a lateral flow immunoassay (LFA) to detect tenofovir (TFV) in urine samples from the TARGET study described in Example 3.

Using urine samples from the TARGET study described above, a lateral flow assay (LFA) for tenofovir was developed as previously described (Koczula K M, Gallotta A., Essays Biochem, 60: 111-120 (2016)). The LFA test strip components include a sample pad onto which the test sample (e.g., urine) is applied; a conjugate pad coated with tenofovir-specific antibodies conjugated to colloidal gold nanoparticles; a nitrocellulose membrane striped with a test line consisting of a tenofovir antigen and a control line consisting of anti-rabbit antibody; and an absorbent pad designed to draw the sample across the reaction membrane by capillary action. Further details regarding the assay design are described in, e.g., Gandhi et al., AIDS, 34: 255-260 (2020).

To evaluate the performance of the LFA, urine samples were aliquoted for measurement by both LC-MS/MS and the LFA. For the LC-MS/MS-based method, tenofovir was separated from one thousand-times diluted urine via reverse-phase high performance LC and quantified by MS/MS using electrospray positive ionization in multiple reaction monitoring mode (TFV, 287.9/175.9 m/z (Q1/Q3)) as previously described (Gandhi et al., EClinical Medicine (Published by The Lancet). Available at: doiorg/101016/jeclinm201808004 (2018)). The lower limit of quantification (LLOQ) of the LCMS/MS-based assay was 500 ng/ml. For the LFA, two to three drops of urine were applied from the urine sample on to the LFA and, after approximately two minutes, the lines on the LFA window were read.

The sensitivity, specificity, and accuracy of the LFA were calculated and compared with LC-MS/MS by cross-tabulating values above/below the 1,500 ng/ml threshold by the two different assays. As misclassification was very rare, confidence intervals were presented based on exact calculations using the binomial distribution (Agresti A, Kateri M, “Categorical data analysis,” In: Lovric M. (ed), International Encyclopedia of Statistical Science, Berlin, Heidelberg: Springer; 2011).

Of 637 urine samples collected among the participants in the TARGET DOT study, 300 were randomly selected to be tested by both the LFA and the gold standard method of LC-MS/MS for validation. The LFA demonstrated 97% specificity (95% CI 93-99%) and 99% sensitivity (94-100%) compared with LC-MS/MS. The LFA accurately classified 98% of patients who took a dose within 24 hours as adherent.

### Example 5

This example describes a study to examine the relationship between urine tenofovir (TFV) levels and HIV seroconversion and objective adherence metrics in a large pre-exposure prophylaxis (PrEP) demonstration project.

PrEP was provided to 1,085 men who have sex with men (MSM) and 140 transwomen (Grant et al., Lancet Infect Dis, 14: 820-829 (2014)). Urine was collected every 12 weeks and dried blood spots (DBS) were prepared 4 and 8 weeks after PrEP initiation, and then every 12 weeks. DBS assays for TFV-diphosphate (TFV-DP) and FTC-triphosphate (FTC-TP) were analyzed at all visits for participants who seroconverted in the iPrEx open-label extension (iPrEx-OLE) study and in a random subset of those who remained HIV-negative (Grant et al., supra). Hair samples for TFV and FTC were collected every 12 weeks for all who provided opt-in consent and analyzed among seroconverters and a random subset of those who remained HIV-negative (Gandhi et al., Lancet HIV, 3: e521-e528 (2016)). Participants who qualified for the correlation analysis required sample availability from all three biomatrices (urine, DBS, hair) at one or more visits over the duration of iPrEx-OLE (median 72 weeks). Additional urine samples from seroconverters (n=10) were included in the specific analysis looking at the association between urine TFV levels and seroconversion. All individuals in the study provided informed consent, including for sample storage and further testing, and the institutional review board from each study site approved the study.

Any TFV level below the lower limit of quantification was considered negative (<1,000 ng/mL). The upper limit of quantification for the immunoassay was 50,000 ng/mL. Using previously described and validated LC-MS/MS-based methods, TFV-DP and FTC-TP concentration were measured in DBS (Zheng et al., J Pharm Biomed Anal, 122: 16-20 (2016)), and FTC and TFV concentrations were measured in hair (Liu et al., PLoS One, 9: e83736 (2014)).

For the seroconversion analysis, urine TFV concentrations via the immunoassay were compared using Kruskal-Wallis' test among individuals at the seroconversion visit; prior to the seroconversion visit; and those who remained HIV-negative. Receiver operating curves (ROC) were analyzed to identify two urine TFV cut-points and compared with the outcome of future HIV seroconversion. Mixed-effects logistic regression examined the association between the cut-points and HIV seroconversion only in the samples collected prior to the seroconversion visit. Spearman correlation coefficients and scatterplots were examined to assess the relationship between TFV urine concentrations via the immunoassay and both TFV-DP and FTC-TP levels in DBS and TFV and FTC levels in hair for participants with samples from all three biomatrices. The sensitivity and specificity of the urine assay at an undetectable urine TFV level (<1,000 ng/mL) was compared with two levels of inadequate adherence defined by TFV-DP concentrations in DBS: the limit of quantification (<3.5 fmol/punch) and very low adherence (<350 fmol/punch, estimated average weekly adherence of <2 tablets/week) (Grant et al., Lancet Infect Dis, 14: 820-829 (2014)); and Anderson et al., Antimicrob Agents Chemother, 62: e01710-e01717 (2018)). The lower limit of detection for the urine assay (1,000 ng/mL) was selected as the optimal single cut-off based on analysis of ROC curves and prior data examining LC-MS/MS-based methods to quantify TFV levels in urine (Lalley-Chareczko et al., J Acquir Immune Defic Syndr, 79: 173-178 (2018)).

The median urinary TFV level was 15,000 ng/mL in those who remained HIV negative (n=105; interquartile range: 1,000-45,000); 5,500 in those who eventually seroconverted (n=11; interquartile range: 1,000-12,500); and all were undetectable at seroconversion (n=9; P<0.001). Decreasing strata of urine TFV levels were associated with future HIV seroconversion (P=0.03). An undetectable urine TFV was 100% sensitive and 81% specific when compared with an undetectable DBS TFV-diphosphate level and 69% sensitive, but 94% specific when compared with low adherence by DBS (<2 doses/week).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A polyclonal antibody composition comprising a heterogeneous population of mammalian antibodies that specifically bind tenofovir (TFV) or a tenofovir derivative, wherein the heterogeneous population of mammalian antibodies is generated against a compound of formula (I) or formula (II): 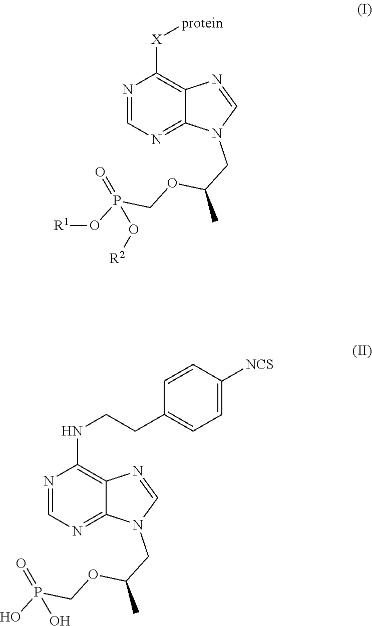
or a pharmaceutically acceptable salt thereof,
wherein:
R¹ and R² are each independently selected from the group consisting of hydrogen, C₁-C₆ alkyl, C₂-C₆ alkenyl, C₂-C₆ alkynyl, aryl, arylalkyl, cyanoalkyl, and —(C₁-C₆-alkylene)-Y—(C₁-C₆ alkyl), wherein Y is selected from —O—, —NH—, —S—, —C(O)NH—, —C(O)O—, —C(O)S—, —OC(O)NH—, —OC(O)O—, and —NHC(O)NH—; and
X is a linker.

2. The composition of claim 1, wherein each of R¹ and R² of the compound of formula (I) is hydrogen.

3. The composition of claim 1, wherein each of R¹ and R² of the compound of formula (I) is —(C₁-C₆-alkylene)-Y—(C₁-C₆ alkyl), wherein Y is —OC(O)O—.

4. The composition of claim 3, wherein each of R¹ and R² of the compound of formula (I) is —CH₂OC(O)OCH(CH₃)₂.

5. The composition of any one of claims 1-4, wherein X of the compound of formula (I) comprises a moiety derived from the reaction of two reactive groups.

6. The composition of claim 5, wherein X of the compound of formula (I) comprises a moiety selected from the group consisting of:

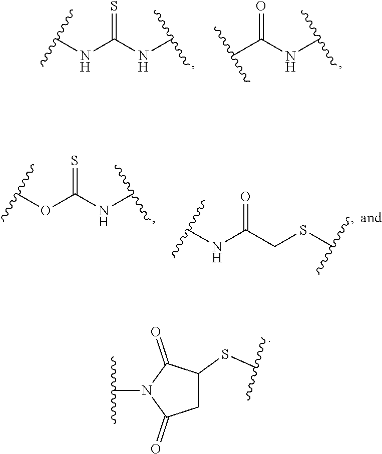 [SMILES: *NC(=S)N* ; *NC(*)=O ; *NC(=S)O* ; *NC(=O)CS* ; *SC1CC(=O)N(*)C1=O]

7. The composition of any one of claims 1-6, wherein X of the compound of formula (I) is: 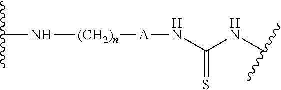
wherein:
n is 1, 2, 3, or 4; and
A is selected from arylene, heteroarylene, cycloalkylene, and heterocyclylene.

8. The composition of claim 7, wherein X of the compound of formula (I) is:

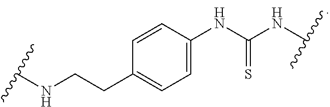 [SMILES: *NCCc1ccc(NC(=S)N*)cc1]

9. The composition of any one of claims 1-8, wherein the protein in the compound of formula (I) is thyroglobulin, albumin, or hemocyanin.

10. The composition of any one of claims 1-9, wherein at least a portion of the heterogeneous population of mammalian antibodies is immobilized on a solid support.

11. The composition of claim 10, wherein the solid support is a microparticle, a test strip, or a sample pad.

12. A solid support for detecting the presence of tenofovir or a tenofovir derivative in a sample, which comprises the polyclonal antibody composition of any one of claims 1-9 immobilized thereon.

13. The solid support of claim 12, which is a microparticle, a test strip, or a sample pad.

14. A method of detecting tenofovir or a tenofovir derivative in a sample obtained from a subject, which method comprises:
(a) contacting a sample obtained from a subject with the solid support of claim 12 or claim 13 under conditions which allow binding of tenofovir or a tenofovir derivative, if present in the sample, to the polyclonal antibody composition, and
(b) detecting binding of tenofovir or a tenofovir derivative bound to the polyclonal antibody composition.

15. The method of claim 14, wherein detecting binding comprises an enzyme-linked immunosorbent assay (ELISA) or a lateral flow immunoassay (LFA).

16. The method of claim 14 or claim 15, wherein the subject is undergoing treatment with tenofovir or a derivative thereof.

17. The method of claim 16, which is repeated two or more times during treatment with tenofovir.

18. The method of any one of claims 14-17, wherein the sample is urine, serum, hair, or saliva.

19. An assay for detecting the presence of tenofovir or a tenofovir derivative in a sample obtained from a subject, which comprises: (i) contacting a biological sample with the polyclonal antibody composition of any one of claims 1-11, wherein the subject is undergoing treatment with tenofovir or a tenofovir derivative; and (ii) detecting the polyclonal antibody composition bound to tenofovir or a tenofovir derivative.

20. The assay of claim 19, wherein the detection comprises an enzyme-linked immunosorbent assay (ELISA) or a lateral flow immunoassay (LFA).

21. The assay of claim 19 or claim 20, wherein the sample is urine, serum, hair, or saliva.

22. Use of a polyclonal antibody composition to detect tenofovir or a tenofovir derivative in a sample obtained from a subject, wherein the polyclonal antibody composition comprises a heterogeneous population of mammalian antibodies that specifically bind tenofovir (TFV) or a tenofovir derivative, and wherein the heterogeneous population of mammalian antibodies is generated against a compound of formula (I) or formula (II): 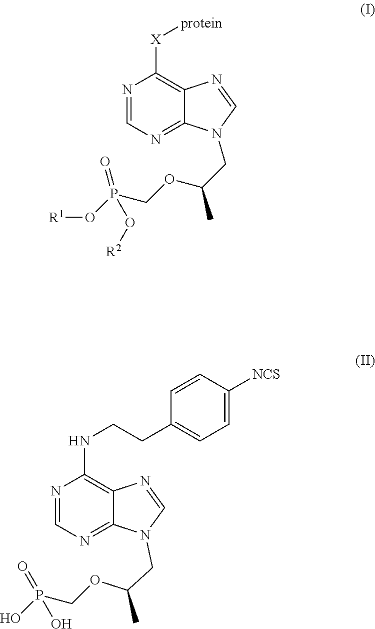
or a pharmaceutically acceptable salt thereof,
wherein:
R1 and R2 are each independently selected from the group consisting of hydrogen, C1-C6 alkyl, C2-C6 alkenyl, C2-C6 alkynyl, aryl, arylalkyl, cyanoalkyl, and —(C1-C6-alkylene)-Y—(C1-C6 alkyl), wherein Y is selected from —O—, —NH—, —S—, —C(O)NH—C(O)O—, —C(O)S—, —OC(O)NH—, —OC(O)O—, and —NHC(O)NH—; and
X is a linker.
